(19) United States
(12) Patent Application Publication
(10) Pub. No.: US 2022/0168301 A1
(43) Pub. Date: Jun. 2, 2022

(54) METHODS FOR TREATING SYMPTOMATIC ORTHOSTATIC HYPOTENSION

(71) Applicant: Cerecor Inc, Rockville, MD (US)

(72) Inventors: Pericles Calias, Melrose, MA (US); Michael Bouchon, Matthews, NC (US); John Boland, Huntersville, NC (US)

(21) Appl. No.: 17/441,246

(22) PCT Filed: Mar. 18, 2020
(86) PCT No.: PCT/US2020/023364

§ 371 Date: Sep. 20, 2021

Related U.S. Application Data
(60) Provisional application No. 62/820,674, filed on Mar. 19, 2019.

(51) Int. Cl. A61K 31/506; A61K 31/165; A61K 31/573; A61K 31/198; A61K 31/4425; A61P 9/02

(52) U.S. Cl. CPC A61K 31/506; A61K 31/165; A61P 9/02; A61K 31/198; A61K 31/4425; A61K 31/573

(57) ABSTRACT

The present disclosure provides a method for treating symptomatic orthostatic hypotension using a potent selective antagonist of N-methyl-D-aspartate receptor subunit 2B (NMDA-GluN2B or NR2B).

### FIELD OF THE INVENTION

The embodiments of the present invention relate to methods for treating symptomatic orthostatic hypotension using a potent selective antagonist of N-methyl-D-aspartate receptor subunit 2B (NMDA-GluN2B or NR2B).

### BACKGROUND OF THE INVENTION

Orthostatic hypotension (OH) is a very common problem, particularly in the frail elderly. It is due to a variety of medical conditions, such as intravascular volume depletion, severe anemia, use of antihypertensive therapies, physical deconditioning, and various underlying diseases. The condition may resolve once the underlying cause is treated; however, for some, it can be a chronic condition.

Neurogenic orthostatic hypotension (nOH) is a much less common and chronic condition. nOH is the result of a failure to increase sympathetic vasomotor nerve outflow and an inability to raise peripheral vascular resistance on standing.^{1,2} nOH is defined by low blood pressure that occurs shortly after sitting or standing up. When blood pressure drops, symptoms can include dizziness, lightheadedness, feeling faint, weakness, blurry vision, head and neck pain, fatigue and syncope. Symptoms can be severe, especially at the start of each day and these symptoms are often associated with an increased risk for falls and injury.^{3,4,5} The goal of treatment of nOH is to reduce symptom burden, prolong standing time, and improve physical capabilities. The steps in management include: (i) removing aggravating factors; (ii) implementing non-pharmacological measures; and (iii) drug therapies. However, up to 70% patients with nOH also have supine hypertension, which poses a therapeutic challenge. Increasing blood pressure in the upright position can worsen hypertension when supine. Therefore, treatment of nOH requires careful consideration of the potential risks and benefits.

There are no long-term studies showing the impact of treatment on survival, falls or quality of life and, importantly, current pharmacologic treatments of symptomatic OH have significant side effects that limit their usefulness. For example, fludrocortisone (Florinef; 9α-fluorocortisol), a synthetic mineralocorticoid that is sometimes used off-label to treat symptomatic OH, increases blood pressure via sodium and water retention, thereby increasing circulating blood volume. However, fludrocortisone commonly causes supine hypertension, and can cause or aggravate renal failure. In an elderly population, concern for fluid overload leading to congestive heart failure needs to be considered.⁶ Long-term use exacerbates supine hypertension and produces end-organ target damage.⁷

ProAmatine® (midodrine hydrochloride), a selective α1-adrenoreceptor agonist that increases vascular resistance and blood pressure, was the first drug approved by the FDA for the treatment of symptomatic orthostatic hypotension in 1996.⁸ Supine hypertension is the main safety concern, and the drug carries a boxed warning that “[b]ecause ProAmatine® can cause marked elevation of supine blood pressure, it should be used in patients whose lives are considerably impaired despite standard clinical care” and cautions that “clinical benefits of ProAmatine®, principally improved ability to carry out activities of daily living, have not been verified.”⁹

Norther® (Droxidopa; L-threo-3,4-dihydroxyphenyl-serine or L-DOPS) is a synthetic catechol-amino acid that, after oral administration, is converted to the naturally-occurring sympathetic neurotransmitter norepinephrine, resulting in an increase in blood pressure. Although Phase III clinical trials showed that droxidopa treatment led to significant improvement in symptoms of nOH (dizziness, lightheadedness or feeling about to faint) with an associated increase in standing systolic blood pressure, the effectiveness has not been demonstrated beyond two weeks.^{10, 11} Further, Northera® also carries a boxed warning that it “can cause supine hypertension and may increase cardiovascular risk if supine hypertension is not well-managed.”¹²

Accordingly, there is a significant need for a long-term therapeutic treatment of symptomatic orthostatic hypotension that is well tolerated and effective.

### BRIEF SUMMARY OF THE INVENTION

The present invention is related to methods for treating symptomatic OH and symptoms thereof using a potent selective antagonist of N-methyl-D-aspartate receptor subunit 2B (NMDA-GluN2B or NR2B). Administration of a pharmaceutical composition comprising an effective amount of a NR2B antagonist to a human patient in need thereof, results in one or more of: (a) an increase in the patient's seated systolic blood pressure; (b) an increase in the patient's standing time; and (c) a decrease in dizziness or lightheadedness experienced by the patient. In some embodiments, the patient suffers from multiple system atrophy, pure autonomic failure, or Parkinson's disease.

In some embodiments, the method for treating symptomatic OH and the symptoms thereof comprises administering to the patient a pharmaceutical composition comprising an effective amount of Compound (I)

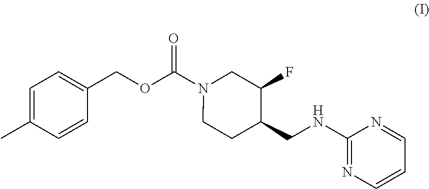 [SMILES: Cc1ccc(COC(=O)N2CC[C@H](CNc3ncccn3)[C@H](F)C2)cc1]

(also known as CERC-301 or MK-0657). In an alternate embodiment, the crystalline form of Compound (I) is administered to the patient for treating symptomatic OH and the symptoms thereof.

In some embodiments, the effective amount of Compound (I) is an amount ranging from about 0.1 mg/day to about 100 mg/day. In other embodiments, the effective amount of Compound (I) is an amount ranging from about 0.5 mg/day to about 50 mg/day. In alternate embodiments, the effective amount of Compound (I) is an amount ranging from about 5.0 mg/day to about 20 mg/day. In yet another alternate embodiments, the effective amount of Compound (I) is 8.0 mg/day, 12 mg/day, 16 mg/day, or 20 mg/day.

In some embodiments, the NR2B antagonist is administered with an agent selected from an α1-adrenoceptor agonist, an α-2 adrenergic receptor antagonist, a corticosteroid, a norepinephrine precursor, and a cholinesterase inhibitor, or a combination thereof. In yet other embodiments, the compound or composition is administered with midodrine, fludrocortisone acetate, droxidopa or pyridostigmine, or, in each case, a pharmaceutically-acceptable salt thereof.

In some embodiments, the patient is afflicted with nOH.

According to the methods of the present invention, the NR2B antagonist administered is well tolerated and provides a long-term effective therapeutic treatment of symptomatic OH and the symptoms thereof. Thus, the patient is treated for a period of at least 12 weeks. In many cases, long-term administration is for at least 4, 5, 6, 7, 8, 9 months or more.

Other implementations are also described and recited herein.

### BRIEF DESCRIPTION OF THE DRAWINGS

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1 depicts the effects of four different doses of CERC-301 on systolic blood pressure over a period of 12 hours in normal subjects.

FIG. 2 provides a line graph showing the pharmacokinetics of four doses of CERC-301 over a six hour period of time: 8 mg (red line); 12 mg (green line); 16 mg (yellow line); and 20 mg (blue line). Error bars represent standard deviation.

FIG. 3 provides a line graph depicting the systolic blood pressure prior to the orthostatic challenge test (pre-OST) with the patient seated: Placebo (navy line; n=12); 8 mg (red line, n=13); 12 mg (green line; n=10); 16 mg (yellow line; n=10); and 20 mg (blue line; n=8). Error bars represent standard deviation.

FIG. 4 provides a line graph depicting the diastolic blood pressure pre-OST with the patient seated: Placebo (navy line; n=12); 8 mg (red line, n=13); 12 mg (green line; n=10); 16 mg (yellow line; n=10); and 20 mg (blue line; n=8). Error bars represent standard deviation.

FIG. 5 provides a line graph depicting the systolic blood pressure after the patient was standing 1 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 6 provides a line graph depicting the diastolic blood pressure after the patient was standing 1 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 7 provides a line graph depicting the systolic blood pressure after the patient was standing 3 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 8 provides a line graph depicting the diastolic blood pressure after the patient was standing 3 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 9 provides a line graph depicting the systolic blood pressure after the patient was standing 5 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 10 provides a line graph depicting the diastolic blood pressure after the patient was standing 5 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 11 provides a line graph depicting the heart rate pre-OST with the patient seated: Placebo (navy line; n=12); 8 mg (red line, n=13); 12 mg (green line; n=10); 16 mg (yellow line; n=10); and 20 mg (blue line; n=8). Error bars represent standard deviation.

FIG. 12 provides a line graph depicting the heart rate after the patient was standing 1 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 13 provides a line graph depicting the heart rate after the patient was standing 3 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 14 provides a line graph depicting the heart rate after the patient was standing 5 min: Placebo (navy line; n=12); 8 mg (red line, n=13); 12 mg (green line; n=10); 16 mg (yellow line; n=10); and 20 mg (blue line; n=8). Error bars represent standard deviation.

FIG. 15 provides a line graph depicting the orthostatic challenge test (OST) profile of systolic blood pressure (SBP) pre-dose: Placebo (navy line; n=12); 8 mg (red line, n=13); 12 mg (green line; n=10); 16 mg (yellow line; n=10); and 20 mg (blue line; n=8).

FIG. 16 provides a line graph depicting the OST profile of SBP at 1 hour post-dose: Placebo (navy line; n=12); 8 mg (red line, n=13); 12 mg (green line; n=10); 16 mg (yellow line; n=10); and 20 mg (blue line; n=8).

FIG. 17 provides a line graph depicting the OST profile of SBP at 2 hours post-dose: Placebo (navy line; n=12); 8 mg (red line, n=13); 12 mg (green line; n=10); 16 mg (yellow line; n=10); and 20 mg (blue line; n=8).

FIG. 18 provides a line graph depicting the OST profile of SBP at 3 hours post-dose: Placebo (navy line; n=12); 8 mg (red line, n=13); 12 mg (green line; n=10); 16 mg (yellow line; n=10); and 20 mg (blue line; n=8).

FIG. 19 provides a line graph depicting the OST profile of SBP at 4 hours post-dose: Placebo (navy line; n=12); 8 mg (red line, n=13); 12 mg (green line; n=10); 16 mg (yellow line; n=10); and 20 mg (blue line; n=8).

FIG. 20 provides a line graph depicting the OST profile of SBP at 6 hours post-dose: Placebo (navy line; n=12); 8 mg (red line, n=13); 12 mg (green line; n=10); 16 mg (yellow line; n=10); and 20 mg (blue line; n=8).

### DETAILED DESCRIPTION OF THE INVENTION

It is to be appreciated that certain aspects, modes, embodiments, variations and features of the invention are described below in various levels of detail in order to provide a substantial understanding of the present invention.

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions or processes are described as using specific materials or an order of individual steps, it is appreciated that materials or steps may be interchangeable such that the description of the invention may include multiple parts or steps arranged in many ways as is readily appreciated by one of skill in the art.

### Definition

The definitions of certain terms as used in this specification and the appended claims are provided below. Unless defined otherwise, all technical and scientific terms used herein generally have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex. The terms “subject” and “patient” are used interchangeably herein.

As used herein, the terms “effective amount” and “therapeutically-effective amount” include an amount sufficient to prevent or ameliorate a manifestation of disease or medical condition, such as neurogenic orthostatic hypotension. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treating” or “treatment” or “to treat” or “alleviating” or “to alleviate” refer to therapeutic measures that cure, slow down, lessen symptoms of, and/or halt progression of a diagnosed disease.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

### Symptomatic Orthostatic Hypotension

Systolic blood pressure is transiently and minimally decreased in healthy individuals upon standing. Normal physiologic feedback mechanisms work through neutrally mediated pathways to maintain the standing blood pressure, and thus maintain adequate cerebral perfusion. The compensatory mechanisms that regulate blood pressure upon standing are dysfunctional in subjects with orthostatic hypotension (OH), a condition that may lead to inadequate cerebral perfusion with accompanying symptoms of syncope, dizziness or lightheadedness, unsteadiness, and blurred or impaired vision, among other symptoms.

The autonomic nervous system has a central role in the regulation of blood pressure. Primary Autonomic Failure is manifested in a variety of syndromes. OH is a usual presenting symptom. Primary Autonomic Failure may be the primary diagnosis, and classifications include pure or progressive autonomic failure (PAF), also called idiopathic orthostatic hypotension (Bradbury-Eggleston syndrome) and autonomic failure with multiple system atrophy (Shy-Drager syndrome) and also Parkinson's disease.

Neurogenic orthostatic hypotension (nOH) is a sub-type of OH. With nOH and other forms of orthostatic hypotension, patients experience the same symptoms but for different reasons. nOH occurs in people with an existing neurologic disease, such as, Parkinson's disease, multiple system atrophy (MSA), pure autonomic failure (PAF), diabetic neuropathy, and dopamine beta hydroxylase (DBH) deficiency.¹³ In other forms of OH, symptoms can be caused by many different factors including dehydration, cardiovascular diseases, and certain medications, such as medication for Parkinson's disease or hypertension.

Regardless of the type of orthostatic hypotension, symptoms usually occur shortly after sitting or standing up. When blood pressure drops, symptoms can include: dizziness, lightheadedness, feeling faint, weakness, blurry vision, head and neck pain, fatigue and syncope. Symptoms can be severe, especially at the start of each day and these symptoms are often associated with an increased risk for falls and injury.^{14,15,16} Symptomatic orthostatic hypotension can make the lives of patients and the people caring for them more difficult, by causing disabling symptoms that make it harder to stand up and walk around, causing fear of falls and injury, and reducing independence.

According to consensus guidelines, OH is defined as a sustained fall of systolic blood pressure by at least 20 mm Hg or diastolic blood pressure by 10 mm Hg within 3 min of standing or head-up tilt.¹⁷ Since the magnitude of blood pressure drop also depends on baseline values, it was suggested that a drop of 30 mm Hg may be a more appropriate criterion for OH in patients with supine hypertension.¹⁸ Blood pressure is a clinical measure and the patients are not necessarily aware of its dysregulation. The prevalence of OH increases with age and is commonly associated with neurodegenerative diseases including Parkinson's disease, dementia with Lewy bodies, multiple system atrophy, and pure autonomic failure. In the general aged population, the prevalence rates of OH range between 5% and 30%.¹⁹

nOH can arise from primary neurodegenerative disorders or can be secondary to systemic conditions that influence peripheral nerve function.²⁰ Parkinson's disease, dementia with Lewy bodies, multiple system atrophy, and pure autonomic failure belong to a category of neurodegenerative disorders known as a-synucleinopathies due to their cellular hallmark feature that is α-synuclein inclusion pathology.²¹ The prevalence of nOH in Parkinson's disease ranges from 16 to 58%.^{22,23} Likewise, in dementia with Lewy bodies symptomatic, OH is found in 30-50% of the patients.^{24,25,26} Both Parkinson's disease and dementia with Lewy bodies show markedly decreased myocardial [¹²³I]-metaiodobenzylguanidine uptake indicating severe impairment of the cardiac sympathetic innervations.^{27′28} nOH symptoms are present in more than two-thirds of all multiple system atrophy patients.²⁹ Pure autonomic failure is characterized by severe nOH-associated with insidious onset, slow progression, modest gastrointestinal impairment, marked supine hypertension and often very low plasma noradrenalin levels representing a characteristic prototype of nOH.³⁰

### Treatment of Symptomatic Orthostatic Hypotension

Symptomatic OH can be a severely disabling condition, which may seriously interfere with the quality of life of afflicted subjects. Although consensus guidelines for the treatment of symptomatic OH are lacking, reviews of available treatments are available.^{31,32} The goal of treatment of symptomatic OH is to reduce symptom burden, prolong standing time, and improve physical capabilities. The steps in management include: (i) removing aggravating factors; (ii) implementing non-pharmacological measures; and (iii) drug therapies. However, up to 70% patients with symptomatic OH also have supine hypertension, which poses a therapeutic challenge. Increasing blood pressure in the upright position can worsen hypertension when supine. Therefore, treatment of symptomatic OH requires careful consideration of the potential risks and benefits.

Currently available therapeutic options provide some symptomatic relief in a subset of subjects but are relatively ineffective and are often accompanied by severe side effects that limit their usefulness. Support garments (tight-fitting leotard) may prove useful in some subjects but is difficult to don without family or nursing assistance, especially for older subjects. Droxidopa, midodrine, fludrocortisone, methylphenidate, ephedrine, indomethacin, and dihydroergotamine are among some of the pharmacological interventions that have been used to treat symptomatic OH, although only droxidopa and midodrine are specifically approved for this indication.

Currently available therapeutic options have significant limitations. For example, fludrocortisone (9α-fluorocortisol; Florinef), a synthetic mineralocorticoid that is sometimes used off-label to treat symptomatic OH that increases blood pressure via sodium and water retention, thereby increasing circulating blood volume, is known to commonly causes supine hypertension, and can cause or aggravate renal failure. In an elderly population, concern for fluid overload leading to congestive heart failure needs to be considered.³³ Long-term use exacerbates supine hypertension and produces end-organ target damage.³⁴

ProAmatine® (midodrine hydrochloride) is a selective al-adrenoreceptor agonist that increases vascular resistance and blood pressure. It was the first drug approved by the FDA for the treatment of symptomatic orthostatic hypotension back in 1996.³⁵ Supine hypertension is the main safety concern, and the drug carries a boxed warning. Indeed, the package insert carries a warning that “[b]ecause ProAmatine® can cause marked elevation of supine blood pressure, it should be used in patients whose lives are considerably impaired despite standard clinical care” and cautions that “clinical benefits of ProAmatine®, principally improved ability to carry out activities of daily living, have not been verified.”³⁶

Northea® (Droxidopa; L-threo-3,4-dihydroxyphenyl-serine or L-DOPS) is a synthetic catechol-amino acid that, after oral administration, is converted to the naturally-occurring sympathetic neurotransmitter norepinephrine, which induces an increase in blood pressure. Although Phase III clinical trials showed that droxidopa treatment led to significant improvement in symptoms of nOH (dizziness, lightheadedness or feeling about to faint) with an associated increase in standing systolic blood pressure, the effectiveness has not been demonstrated beyond two weeks.^{37,38} Further, Northera® also carries a boxed warning that it “can cause supine hypertension and may increase cardiovascular risk if supine hypertension is not well-managed.”³⁹

The limitations of these currently available therapeutic options, and the incapacitating nature and often progressive downhill course of the disease, point to the need for an improved therapeutic alternative. The present invention provides a long-term therapeutic treatment of neurogenic orthostatic hypotension that is well tolerated and effective using a potent selective antagonist of N-methyl-D-aspartate receptor subunit 2B (NMDA-GluN2B or NR2B). NR2B antagonists useful in the methods of the present invention include, but are not limited to, the NR2B antagonists described in U.S. Pat. Nos. 7,053,089; 7,592,360; and 10,202,363, the disclosure of each is incorporated herein by reference in their entirety. In one embodiment of the present invention, the NR2B antagonist is CERC-301 (previously known as MK-0657 and L-001067743), an orally bioavailable N-methyl-D-aspartate (NMDA) receptor antagonist with selectivity for the NR2B subunit originally developed by Merck.

### Pre-Clinical Studies with Cerc-301

GluN2BR antagonists have been demonstrated to stimulate renal sympathetic nerve activity and to have marked pressor effects that are blocked by β1 adrenoceptors blockers. CERC-301 has been shown to increase heart rate and blood pressure in rats and monkeys. These effects are blocked by β1 and α1 adrenoceptors blockers, respectively.

Pharmacokinetic (PK) studies in rats and monkeys suggested CERC-301 is rapidly absorbed after oral administration, is highly metabolized via multiple cytochrome P450 (CYP450) enzymes, and is excreted exclusively as metabolites via both the biliary and renal routes. Studies in human, rat, and monkey microsomes and hepatocytes suggest similar elimination across species. Data from nonclinical studies identified one active metabolite of CERC-301.

The single and repeat-dose toxicology studies in rats and monkeys suggested that potential adverse effects of CERC-301 in human subjects would involve the nervous system and most likely would reflect the drug's intended pharmacodynamic activity.

Unfortunately, preclinical studies do not offer any information on the therapeutic potential of drugs for treatment of nOH. The lack of accepted animal models for nOH means that clinical studies are the only venue for assessing the therapeutic potential of NR2B antagonists for treatment of nOH.

### Clinical Studies with Cerc-301

Merck completed two Phase I studies of CERC-301 in healthy males (N=56) and one Phase I study in healthy elderly males and females (N=27). Two additional Phase IB studies were completed in subjects with moderate PD (N=38). A Phase IB study in subjects (N=5) with treatment-resistant depression (TRD) was conducted under a separate Investigational New Drug application (IND) as an Investigator initiated study by the National Institute of Mental Health (NIMH). While only five subjects participated in the study, antidepressant effects of CERC-301 were observed.⁴⁰ In another study, CERC-301 was investigated for improvement in motor symptoms in a preclinical model of Parkinson's disease using a randomized, double-blind, double-dummy, placebo-controlled, 3-period crossover study was conducted in patients with moderate Parkinson's disease.⁴¹ Patients (N=16) received single oral doses of 7 mg CERC-301, carbidopa/levodopa 25/250 mg as a positive control, and placebo, after which motor function was serially evaluated by means of the Unified Parkinson's Disease Rating Scale—Motor Examination (UPDRS-ME). CERC-301 administration showed no improvement compared with placebo (P=0.110) despite exceeding the target plasma concentration of 400 nM. Although the administration of CERC-301 was generally well tolerated, it was associated with increases in systolic and diastolic blood pressure relative to placebo. It was thus concluded that the results did not support ongoing clinical development of MK-0657 as a novel monotherapy for Parkinson's disease.⁴² Cerecor Inc. has completed two Phase 2 studies in major depressive disorder (MDD) (N=137 and N=115).

Given that symptomatic OH results from an inability to regulate blood pressure in response to orthostatic challenge, it is possible that the pressor activity of CERC-301 could have utility in the treatment of symptomatic OH and indications such as nOH. Based on the observation that CERC-301 appears to increase blood pressure through an increase in sympathetic outflow (a different pressor mechanism from that of the currently approved adrenergic agonists droxidopa and midodrine), it was hypothesized that CERC-301 may have the potential to synergize with these therapies for the treatment of symptomatic OH.

### EXAMPLES

The invention now being generally described, it will be more readily understood by reference to the following examples which are included merely for purposes of illustration of certain aspects and embodiments of the present invention, and are not intended to limit the invention.

### Example 1

### A Randomized, Double-Blind, Placebo-Controlled, Parallel-Group, Three-Part Safety, Pharmacokinetic, and Pharmacodynamic Study of CERC-301 in Healthy Subjects

The effects of CERC-301 were assessed in a randomized, double-blind, placebo-controlled, parallel-group, three-part safety, pharmacokinetic, and pharmacodynamic study in healthy human subjects.

One of the primary objective of the study was to investigative the dose-response relationship between CERC-301 and pharmacodynamic (PD) effects on blood pressure [BP] in healthy subjects. Secondary objectives included the investigation of the safety and tolerability of CERC-301 over 7 days of once-daily administration and the investigation of a single dose and 7-day repeated dose pharmacokinetic (PK) profiles of CERC-301, and to explore sub-group (age or gender) effects on other safety parameters, such as adverse events (AEs).

### 1 Methodology

The study was a randomized, double-blind, placebo-controlled parallel-group, three-part, repeated dose in-patient study investigating the safety, tolerability, PK, and PD of CERC-301 in healthy subjects.

Part 1: After the screening period, healthy, young male and female subjects were randomly assigned to dose groups as shown in Table 1. Randomized subjects participated in a time-matched baseline BP monitoring day (Day −1), once-daily dosing with study drug during which safety, PK, and PD assessments were performed on Days 1-7, discharge on Day 8, a follow-up period for PK and safety on Days 9-11, and end-of-study visit on Day 16. Each parallel dose group was balanced for gender (approximately equal number of males and females in each group).

TABLE 1
Part 1 Parallel Dose Groups (Dosing Days 1-7)

| Age | Group* | Study Drug | Dosing Regimen |
|---|---|---|---|
| Young | 1 (N = 6) | CERC-301 | Two 4-mg tablets + one placebo |
| | | 8 mg | tablet once-daily with food |
| Young | 2 (N = 6) | CERC-301 | Three 4-mg tablets once-daily |
| | | 12 mg | with food |
| Young | 3 (N = 4) | Placebo | Three placebo tablets once-daily |
| | | | with food |

*Randomization was stratified such that three males and three females were planned to be randomized to each dose group.
Note:
On Day -1 (time-matched baseline day), all subjects received placebo tablets in the fed state.

Part 1 Safety Review: Safety data from Part 1 were reviewed and a decision was made to conduct Part 2 as planned (Table 2) and not according to an alternative dosing regimen (repeat a previous dose level or any combination of changing the dose level, dosing interval, or fed state).

Part 2: After the screening period, healthy, young and intermediate age male and female subjects were randomly assigned to dose groups as shown in Table 2. Randomized subjects participated in a time-matched baseline BP monitoring day (Day −1), once-daily dosing with study drug during which safety, PK, and PD assessments were performed on Days 1-7, discharge on Day 8, a follow-up period for PK and safety on Days 9-11, and end-of-study visit on Day 16. Each parallel dose group was balanced for gender (approximately equal number of males and females in each group).

Part 2 Safety Review: Safety data from Part 2 was reviewed and a decision was made to conduct Part 3 as planned (Table 3) and not according to an alternative dosing regimen (repeat a previous dose level or any combination of changing the dose level, dosing interval, or fed state).

Part 3: After the screening period, healthy, young and elderly male and female subjects were randomly assigned to dose groups as shown in Table 3.

TABLE 2
Part 2 Parallel Dose Groups (Dosing Days 1-7)

| Age | Group* | Study Drugs§ | Dosing Regimen** |
|---|---|---|---|
| Young | 4 (N = 6) | CERC-301 | Four 4-mg tablets |
| | | 16 mg | once-daily with food |
| Young | 5 (N = 2) | Placebo | Four placebo tablets |
| | | | once-daily with food |
| Intermediate | 6 (N = 6) | CERC-301 | Three 4-mg tablets |
| | | 12 mg | once-daily with food |
| Intermediate | 7 (N = 2) | Placebo | Three placebo tablets |
| | | | once-daily with food |

*Randomization was stratified such that an equal number of males and females were planned to be randomized to each dose group.
**The dosing regimen (dosing interval/fed state) could have been changed based on the Part 1 safety review.
§The planned dose level could have been changed based on the outcome of the Part 1 safety review. The maximum dose in young subjects was 16 mg and the maximum dose in the intermediate age subjects was 12 mg.
Note:
On Day -1 (time-matched baseline day), all subjects received placebo tablets in the fed state.

TABLE 3
Part 3 Parallel Dose Groups (Dosing Days 1-7)

| Age | Group* | Study Drugs§ | Dosing Regimen** |
|---|---|---|---|
| Young | 8 (N = 6) | CERC-301 | Five 4-mg tablets once-daily |
| | | 20 mg | with food |
| Young | 9 (N = 2) | Placebo | Five placebo tablets |
| | | | once-daily with food |
| Elderly | 10 (N = 6) | CERC-301 | Three 4-mg tablets |
| | | 12 mg | once-daily with food |
| Elderly | 10 (N = 2) | Placebo | Three placebo tablets |
| | | | once-daily with food |

*Randomization was stratified such that an equal number of males and females were planned to be randomized to each dose group.
**The dosing regimen (dosing interval/fed state) could have been changed based on the Part 2 safety review.
§The planned dose level could have been changed based on the outcome of the Part 2 safety review. The maximum dose in young subjects was 20 mg and the maximum dose in the elderly age subjects was 12 mg.
Note:
On Day -1 (time-matched baseline day), all subjects received placebo tablets in the fed state.

Randomized subjects participated in a time-matched baseline BP monitoring day (Day −1), once-daily dosing with study drug during which safety, PK, and PD assessments were performed on Days 1-7, discharge on Day 8, a follow-up period for PK and safety on Days 9-11, and end-of-study visit on Day 16. Each parallel dose group was balanced for gender (approximately equal number of males and females in each group).

Part 3 Safety Review: Safety data from Part 3 was reviewed and a decision was made to stop the study, or to repeat a previous regimen or study a new CERC-301 dosing regimen (any combination of changing the dose level, dosing interval, or fed state).

### 2 Number of Subjects

It was anticipated that 48 subjects at a single investigative site would be enrolled, with the option to add or subtract dose groups based on safety review. Part 1 was to include 16 healthy, young male and female subjects; Part 2 was to include 16 healthy subjects, including eight healthy, young male and female subjects, and eight healthy, intermediate age male and female subjects; and Part 3 was to include 16 healthy subjects, including eight healthy, young male and female subjects, and eight healthy, elderly male and female subjects. Forty-eight subjects were enrolled and dosed, including 32 young subjects (24 received CERC-301 and eight received placebo), eight intermediate age subjects (six received CERC-301 and two received placebo), and eight elderly subjects (six received CERC-301 and two received placebo); 42 subjects completed the entire study.

### 3 Diagnosis and Main Criteria for Inclusion

Healthy adult male and female subjects without clinically significant history or evidence of cardiovascular, respiratory, hepatic, renal, gastrointestinal, endocrine, neurological, immunological, or psychiatric disorder(s); young subjects were between 18 to 45 years of age, inclusive, intermediate age subjects were 46 to 64 years of age, inclusive, and elderly subjects were 65 years of age at screening; BMI 18.5 to 30.0 kg/m², inclusive at screening.

### 4 Test Product, Dose and Mode of Administration

CERC-301 immediate release 4 mg tablets, white to off white convex tablets; batch number 130064 and matching CERC-301 placebo tablets.

### 5 Duration of Treatment

Subjects received 8, 12, 16 or 20 mg CERC-301 or matching placebo once-daily for 7 days.

### 6 Endpoints for Evaluation

### 6.1 Pharmacodynamics (PD):

PD endpoints included the following determined from plasma CERC-301 concentration data:
24-hour Ambulatory Blood Pressure Monitoring (ABPM)
   Observed values and time matched change from baseline (pre-dose) of 24-hour ABPM
   Mean 24 hours, Sleep and Awake systolic and diastolic BPs.
   Mean Arterial Pressure (MAP), pulse pressure, and heart rate.
Observed values and change from baseline of sub-scale scores and Total Mood Disturbance (TMD) scores from Profiles of Mood States (POMS)

### 6.2 Pharmacokinetics (PK):

PK endpoints included the following determined from plasma CERC-301 concentration data:
Day 1 parameters include Cₘₐₓ, Tₘₐₓ, AUC₀₋₂₄, AUC_{0-inf}, AUC_{%extrap}, CL/F, Vz/F, and t_{1/2}
Day 7 parameters include Cₘₐₓ, Tₘₐₓ, AUC₀₋₂₄, AUC_{0-inf}, AUC_{%extrap}, CL/F, Vss/F, t_{1/2}, and C_{avg}
Trough concentrations will be reported for Days 2-8
Accumulation after 7-days of dosing will be investigated by calculating:
   Observed Accumulation Ratio: AUCo-24 (Day 7)/AUC₀₋₂₄ (Day 1)
   Steady State Accumulation Ratio: AUC₀₋₂₄ (Day 7)/AUC₀₋ᵢₙₜ (Day 1)
   Cₘₐₓ ratio: Cₘₐₓ (Day 7)/Cₘₐₓ (Day 1)

### 7 Safety

The safety variables evaluated included treatment-emergent adverse events (TEAE), vital signs, clinical safety laboratories (chemistry, hematology, urinalysis), electrocardiograms (ECGs), physical examinations, Clinician Administered Dissociated States Scale (CADSS) and Columbia-Suicide Severity Rating Scale (C-SSRS).

### 8 Pharmacodynamic Results

8.1 24-hour, Daytime, and Nighttime Ambulatory Blood Pressure Monitoring (ABPM):

Systolic Blood Pressure (SBP): Average 24-hour and daytime SBP increased by approximately 3 to 6 mm Hg for 8, 12 and 16 mg CERC-301 doses compared to placebo and by approximately 9 to 15 mm Hg, on average, for 20 mg CERC-301 compared to placebo. The effects on average nighttime SBP were less apparent and somewhat inconsistent, with little effect being observed for the 8 and 12 mg CERC-301 dose groups. For average nighttime SBP, in the 16 mg CERC-301 dose group, an average increase of approximately 5 to 8 mm Hg was observed on Days 1 and 4, but not Day 7 (2 mm Hg increase), and in the 20 mg CERC-301 dose group an average increase in nighttime SBP of approximately 3 to 7 mm Hg was observed on Days 1, 4, and 7.

Diastolic Blood Pressure (DBP): Average 24-hour and daytime DBP increased by approximately 2 to 5 mm Hg for 8, 12 and 16 mg CERC-301 doses compared to placebo and by approximately 6 to 11 mm Hg, on average, for CERC-301 20 mg compared to placebo. The effects on average nighttime DBP were less apparent and somewhat inconsistent, with little effect being observed for the 8 and 12 mg CERC-301 dose groups. In the 16 and 20 mg CERC-301 dose groups, an average increase in nighttime DBP of approximately 3 to 4 mm Hg was observed on Days 1, 4, and 7.

The BP increasing effect for average 24-hour and daytime SBP and DBP appeared to reach a maximum by Day 4, except for the 20 mg CERC-301 dose which increased further from Day 4 to Day 7.

### 8.2 Average Hourly Change in ABPM:

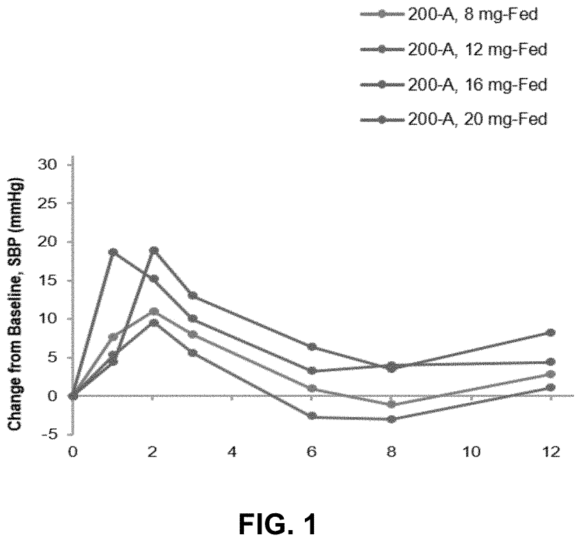

SBP: Mean hourly average change from baseline in ambulatory SBP was increased after 7 days of CERC-301 dosing compared to placebo. The effect of CERC-301 on SBP was most pronounced from 0 to 6 hours postdose for the 8, 12, and 16 mg CERC-301 dose groups and was most pronounced from 0 to 12 hours postdose in the 20 mg CERC-301 dose group. The effect of CERC-301 on increasing SBP was greatest with the 20 mg CERC-301 dose and was still evident (mean hourly average SBP change of approximately 14 mm Hg) at 24 hours postdose. As depicted in FIG. 1, CERC-301 produced a rapid, durable, dose-dependent increases in systolic blood pressure over the first 12 hours.

The maximum average hourly daytime ambulatory SBP was observed between 2 to 6 hours postdose for all CERC-301 doses, with most of the maximum values being 10 to 12 mm Hg on Days 1, 4 and 7 for the 8, 12 and 16 mg CERC-301 doses. The maximum average hourly daytime ambulatory SBP was approximately 16, 21 and 24 mm Hg for the 20 mg CERC-301 dose on Days 1, 4, and 7, respectively. The maximum average hourly nighttime ambulatory SBP was observed between 15 to 20 hours postdose.

DBP: Mean hourly average change from baseline in ambulatory DBP was increased after 7 days of CERC-301 dosing compared to placebo. The effect of CERC-301 on DBP was most pronounced from 0 to 6 hours postdose for the 8, 12, and 16 mg CERC-301 dose groups and was most pronounced from 0 to 12 hours postdose in the 20 mg CERC-301 dose group. The effect of CERC-301 on increasing DBP was greatest with the 20 mg CERC-301 dose and was still evident (mean hourly average DBP change of approximately 10 mm Hg) at 24 hours postdose.

The maximum average hourly daytime ambulatory DBP was observed between 2 to 4 hours postdose for the 8, 12 and 16 mg CERC-301 doses and between 2 to 8 hours postdose for the 20 mg CERC-301 dose. The maximum values were approximately 5 to 12 mm Hg on Days 1, 4 and 7 for the 8, 12 and 16 mg CERC-301 doses. The maximum average hourly daytime ambulatory DBP was approximately 15, 15 and 19 mm Hg for the 20 mg CERC-301 dose on Days 1, 4, and 7, respectively. The maximum average hourly nighttime ambulatory DBP was also observed between 15 to 21 hours postdose.

### 9 Pharmacokinetic Results

### 9.1 Young Subjects

Median tₘₐₓ values ranged from 1.50 to 3.00 hours on Days 1 and 7. On Days 1 and 7, Cₘₐₓ and AUC values increased in an approximately dose-proportional manner from 8 to 20 mg CERC-301. On Day 7, average t_{1/2} ranged from approximately 17 to 21 hours, average apparent oral clearance values ranged from approximately 3 to 4 L/h, and average apparent volume of distribution ranged from approximately 83 to 115 L.

Observed accumulation index was approximately 1.6 to 1.7, on average, and steady-state accumulation index was 1.0 to 1.1, on average. Average Day 7 to Day 1 Cₘₐₓ ratios were 1.2 to 1.4.

### 9.2 Young, Intermediate Age, and Elderly Subgroups

Median tmax values were similar among the three age groups, ranging from 1.50 to 2.50 hours, on average.

On Day 1, average Cₘₐₓ values were similar among all three age groups. On Day 7, average Cₘₐₓ values were approximately 30% higher in the intermediate age and elderly subgroups compared to the young age group.

On Day 1, average AUC values were approximately 25% to 37% higher in the intermediate age group compared to the young age group. On Day 7, average AUC values were approximately 48% to 74% higher in the elderly age group compared to the young age group.

On Day 7, average t_{1/2} ranged from approximately 20 to 28 hours, and was approximately 27% and 38% higher in the intermediate age and elderly age groups, respectively, compared to the young age group. Apparent oral clearance values were approximately 18% and 30%, on average, lower in the intermediate age and elderly groups, respectively, compared to the young age group. Average apparent volume of distribution values were 115 L, 121 L and 111 L in the young age, intermediate age and elderly age subgroups, respectively, on Day 7.

Mean observed accumulation index was 1.65, 1.92 and 2.12 in the young, intermediate age, and elderly age groups, respectively. Mean steady state accumulation index was 1.01, 1.13 and 1.30 in the young, intermediate age, and elderly age groups, respectively. Average Day 7 to Day 1 Cmax ratios were 1.15, 1.41 and 1.53 in the young, intermediate age, and elderly age groups, respectively.

### 9.3 Male and Female Subjects

Median tₘₐₓ values were similar between male and female subjects, ranging from 1.75 to 2.00 hours, on average.

On Day 1 and Day 7, average Cₘₐₓ values were similar in male and female subjects. On Day 1, average AUC values were approximately 6% to 29% higher in female subjects compared to male subjects. On Day 7, average AUC values were approximately 19% to 38% higher in female subjects compared to male subjects.

On Day 7, average t_{1/2} ranged from approximately 21 to 24 hours, and was approximately 16% higher in female subjects compared to male subjects. Apparent oral clearance values were approximately 15%, on average, lower in female subjects compared to male subjects. Average apparent volume of distribution was 108 L in males and 105 L in females on Day 7.

Mean observed accumulation index was 1.72 in males and 1.88 in females. Mean steady state accumulation index was 1.15 in males and 1.10 in females. Average Day 7 to Day 1 Cₘₐₓ ratios were 1.36 in males and 1.38 in females.

### 10 Safety Results

Repeated daily doses of 8, 12, 16, and 20 mg CERC-301 for 7 days were generally well tolerated in these healthy subjects. The most common related AEs were feeling abnormal, headache, feeling of relaxation, elevated mood, dizziness, logorrhea, sedation, visual impairment, energy increased, palpitations, disturbance in attention, hypervigilance, confusional state, oral paresthesia, anxiety, chest pain, depressed mood, diarrhea, and balance disorder, each being reported in at least two subjects receiving CERC-301. Overall, there were no clear-cut dose-related or age-related differences in TEAEs or related AEs.

Overall, there were no clinically significant laboratory abnormalities attributable to CERC-301 except for an elderly female subject receiving 12 mg CERC-301 who experienced mild, reversible alanine aminotransferase and aspartate aminotransferase increases and elevated prolactin in a young male subject receiving 12 mg CERC-301.

There were no clinically significant adverse changes in vital signs, ECGs or physical examination that were attributable to CERC-301.

### 11 Conclusions

The overall purpose of this study was to conduct a thorough evaluation of the effects of single and repeated daily doses of CERC-301 on BP, safety, PK and PD in healthy male and female subjects and in young, intermediate age, and elderly age subgroups.

Pharmacodynamic endpoints included 24-hour ABPM and POMS. With respect to the effects of CERC-301 on ABPM, the average 24-hour and daytime ambulatory SBP and DBP increased with all CERC-301 doses compared to placebo, appearing to reach a maximum by Day 4, except for the 20 mg CERC-301 dose which increased further from Day 4 to Day 7. The effects of CERC-301 on average nighttime SBP and DBP were smaller and less consistently observed. Average 24-hour and daytime SBP increased by approximately 3 to 6 mm Hg for 8, 12 and 16 mg CERC-301 doses compared to placebo and by approximately 9 to 15 mm Hg, on average, for 20 mg CERC-301 compared to placebo. Average 24-hour and daytime DBP increased by approximately 2 to 5 mm Hg for 8, 12 and 16 mg CERC-301 doses compared to placebo and by approximately 6 to 11 mm Hg, on average, for 20 mg CERC-301 compared to placebo.

The effects of CERC-301 on ambulatory BP appeared similar regardless of age subgroup, with no age-dependent differences being observed at the 12 mg dose. The effect of 20 mg CERC-301 on ambulatory SBP appeared slightly more pronounced in females compared to males on Day 7, however, overall, the BP effects of CERC-301 at doses ranging from 8 to 20 mg appeared similar in males and females. With respect to POMS, TMD scores did not reveal any clear differences between CERC-301 doses and placebo and scores.

Pharmacokinetic parameters were determined for CERC-301 using noncompartmental methods based on plasma CERC-301 concentration data following the first and last dose of CERC-301. Following repeated daily doses of CERC-301, steady-state predose plasma CERC-301 concentrations were achieved by study day 5 or 6. Plasma CERC-301 concentrations increased in an approximately dose proportional manner from 8 to 20 mg with steady-state daily CERC-301 dosing. Mean plasma CERC-301 concentrations on Day 7 were approximately 20% to 40% higher from 2 to 24 hours postdose in the intermediate age and elderly subgroups, respectively, compared to the young age subgroup, and were approximately 20% higher from 2 to 24 hours postdose in female subjects compared to male subjects.

With respect to CERC-301 PK parameters, CERC-301 was orally bioavailable with median tmax values ranged from 2.00 to 3.00 hours on Day 1 and from 1.50 to 3.00 hours on Day 7. On Days 1 and 7, Cmax and AUC values increased in an approximately dose-proportional manner over the dose range studied of 8 to 20 mg CERC-301. On Day 7, average t_{1/2} ranged from approximately 17 to 21 hours, average apparent oral clearance values ranged from approximately 3 to 4 L/h, and average apparent volume of distribution ranged from approximately 83 to 115 L. Modest accumulation was observed with seven days of daily CERC-301 dosing as the observed accumulation index was approximately 1.6 to 1.7, on average, and steady-state accumulation index was 1.1, on average. Average Day 7 to Day 1 Cmax ratios were 1.2 to 1.4. Clinically modest differences in PK parameters were observed in intermediate age and elderly subjects compared to young subjects, and in female subjects compared to male subjects. On Day 7, average Cmax values were approximately 30% higher in the intermediate age and elderly subgroups compared to the young age group. On Day 1, average AUC values were approximately 25% to 37% higher in the intermediate age group compared to the young age group and average AUC values were approximately 6% to 29% higher in female subjects compared to male subjects. On Day 7, average AUC values were approximately 48% to 74% higher in the elderly age group compared to the young age group and average AUC values were approximately 19% to 38% higher in female subjects compared to male subjects. On Day 7, apparent oral clearance values were approximately 18% and 30%, on average, lower in the intermediate age and elderly groups, respectively, compared to the young age group and apparent oral clearance values were approximately 15%, on average, lower in female subjects compared to male subjects. On Day 7, average t_{1/2} ranged from approximately 20 to 28 hours, and was approximately 27% and 38% higher in the intermediate age and elderly age groups, respectively, compared to the young age group. On Day 7, average t_{1/2} ranged from approximately 21 to 24 hours, and was approximately 16% higher in female subjects compared to male subjects.

Repeated daily doses of 8, 12, 16, and 20 mg CERC-301 for 7 days were generally well tolerated in these healthy subjects. The most common related AEs were feeling abnormal, headache, feeling of relaxation, elevated mood, dizziness, logorrhea, sedation, visual impairment, energy increased, palpitations, disturbance in attention, hypervigilance, confusional state, oral paresthesia, anxiety, chest pain, depressed mood, diarrhea, and balance disorder, each being reported in at least 2 subjects receiving CERC-301. Overall, there were no clear-cut dose-related or age-related differences in TEAEs or related AEs. There were no clinically significant adverse changes in vital signs, ECGs or physical examination that were attributable to CERC-301.

Given that CERC-301 was well tolerated in healthy subjects across different age groups (young, intermediate, and elderly) and CERC-301-induced dose-related increase in systolic blood pressure and diastolic blood pressure in the present study, CERC-301 appears to have potential for the treatment of patients with symptomatic nOH.

### Example 2

### A Randomized, Double-Blind, Placebo-Controlled, Safety, and Pharmacokinetic Study of CERC-301 in Patients with Symptomatic nOH Associated with Parkinson's Disease

The purpose of this study was to evaluate the safety, tolerability, and PK of single and multiple doses of CERC-301 in patients with symptomatic nOH associated with Parkinson's disease, to explore the effect of single doses of CERC-301 on blood pressure changes during orthostatic challenge compared to placebo, and to explore the effect of single doses of CERC-301 on symptomatic OH compared to placebo.

### 1 Study Design

This study was designed as a randomized, double-blind, placebo-controlled trial in order to distinguish effects of active treatment in an efficient and unbiased manner. As CERC-301 had not previously been tested in subjects with symptomatic OH, a single escalating dose study design was used to ensure safety of the study participants.

Subjects were dosed on five separate occasions, approximately 7-10 days apart, with one of four single escalating doses of CERC-301 or placebo and underwent an orthostatic challenge test to assess the safety, tolerability, effect on blood pressure, and PK. Subjects also completed a symptomatic assessment following the orthostatic challenge. Subjects were enrolled and randomized based on the dosing schedule in Table 4:

TABLE 4
Dosing Schedule

| | Visit 1 | Visit 2 | Visit 3 | Visit 4 | Visit 5 (4:1) |
|---|---|---|---|---|---|
| Arm 1 (n = 5) | Placebo | 8 mg | 12 mg | 16 mg | 20 mg (4:1) |
| Arm 2 (n = 5) | 8 mg | Placebo | 12 mg | 16 mg | 20 mg (4:1) |
| Arm 3 (n = 5) | 8 mg | 12 mg | Placebo | 16 mg | 20 mg (4:1) |
| Arm 4 (n = 5) | 8 mg | 12 mg | 16 mg | Placebo | 20 mg (4:1) |

At Visit 1, subjects were randomized to one of four dose escalating treatment arms. At Visit 5, subjects were randomized again to either 20 mg CERC-301 or placebo (4:1).

### 2 Rationale For Dose Selection and Investigational Drug

In previous studies, a 7 mg CERC-301 dose was the lowest dose to show an effect on blood pressure in study subjects. Doses higher than 20 mg had not been tested to date. A dose range of 8 to 20 mg falls within a 50% or greater receptor occupancy and is well within the toxicologic limits from the 5-week rat and monkey toxicology studies based on AUC and Cₘₐₓ.

Investigational product was administered as CERC-301 4 mg tablets and matching placebo. Study drug was administered orally (P.O) with water approximately 1.5 hours after the patient consumed breakfast.

### 3 Study Population

### 3.1 Number of Patients/Demographics

Approximately 20 patients were planned to complete this study, however following an interim analysis enrolment was closed.
Analyzed:
   Nineteen (19) patients enrolled
   13 patients were randomized and received at least one dose of IP (active or placebo)
   9 patients completed all 5 planned study visits
Demographics
   Average age: 70 years
   Sex: 10 males, 3 females
   Race: 100% White
   Average height: 175 cm
   Average BMI: 23 kg/m²

### 3.2 Inclusion Criteria

Eligible subjects were included if they met the following criteria before being enrolled into the study:
1. Provide written informed consent to participate in the study and understand that they may withdraw their consent at any time without prejudice to their future medical care
2. Male or female and aged 18 years or over
3. Female subjects must be:
   Post-menopausal (amenorrhea for at least 12 consecutive months) or surgically sterile
   -OR-
   Women of childbearing potential (WOCBP) meeting the criteria below:
      Non-lactating and has a negative pregnancy test at screening
      -AND-
      Uses an acceptable double-barrier method of contraception as determined by the Investigator or Sub-Investigator for the duration of the study and 30 days following the last dose of study drug
4. Male subjects must agree to use an acceptable double-barrier method of contraception with their partner as determined by the Investigator or Sub-Investigator for the duration of the study and 30 days following the last dose of study drug
5. Clinical diagnosis of symptomatic nOH associated with Parkinson's disease
6. A documented fall in systolic blood pressure of at least 20 mm Hg, or in diastolic blood pressure of at least 10 mm Hg, within 3 minutes after standing at Screening
7. Persistent symptoms of nOH within the past 14 days
8. Be willing and able to cease taking any medication for nOH for at least 72 hours prior to each other visit throughout the duration of the study

### 3.3 Exclusion Criteria

Subjects meeting any of the following criteria were deemed to be ineligible for the study:
1. Taking long acting anti-hypertensive medication. Short acting antihypertension medication at bedtime is permitted.
2. Patients taking droxidopa with an adequate response to treatment. Patients taking droxidopa may enter the study if they are experiencing persistent symptoms of nOH over the last the past 14 days AND demonstrate a fall in blood pressure of at least SBP 20 mmHg/DBP 10 mmHg within 3 minutes after standing at Screening and remain on a stable dose of droxidopa throughout the duration of the study.
3. Patients currently taking fludrocortisone with an adequate response to treatment. Patients taking fludrocortisone may enter the study if they are experiencing persistent symptoms of nOH over the last the past 14 days AND demonstrate a fall in blood pressure of at least SBP 20 mmHg/DBP 10 mmHg within 3 minutes after standing at Screening and remain on a stable dose of fludrocortisone throughout the duration of the study.
4. Taking moderate and strong CYP3A4 and 2C9 inhibitors and inducers.
5. Have a history of more than moderate alcohol consumption during the last year.
6. Current known or suspected drug abuse.
7. Subjects who are suicidal at screening or have previously expressed suicidal thoughts or tendencies.
8. Subjects who smoke or use tobacco products. Previous smokers or tobacco users are permitted so long as they have not used for at least 1 year.
9. Wheelchair, bedbound, or unable to stand or walk.
10. Women who are pregnant or lactating.
11. Have a history of closed angle glaucoma.
12. Have pre-existing sustained severe hypertension (blood pressure >180/110 mm Hg in the sitting position).
13. Have atrial fibrillation or, in the investigator's opinion, have any other clinically significant cardiac arrhythmia.
14. In the investigator's opinion, have any other significant systemic, hepatic, cardiac, renal or psychiatric illness or cognitive impairment.
15. Have uncontrolled diabetes mellitus or insipidus (hemoglobin [Hb]A1c>7%)
16. Currently known or suspected or a history of cancer within the last 5 years; 2 years for non-metastatic cutaneous squamous cell or basal cell carcinoma or cervical cancer in situ.
17. Have known gastrointestinal illness or other gastrointestinal disorder or procedures that may, in the investigator's opinion, affect the absorption of study drug.
18. In the investigator's opinion, have clinically significant abnormalities on clinical examination or laboratory testing (other than Parkinson's disease and nOH).
19. Abnormal liver function.
20. Currently enrolled or have recently participated in an investigational drug or device study within 30 days or 5 half-lives of the investigational product, whichever is longer.
21. Excessive blood loss or donation of blood (>500 mL) or blood products within 56 days prior to admission (Day −2).
22. Subject has a history of hypersensitivity to NMDA receptor modulators (e.g., dextromethorphan, ketamine, amantadine, memantine) or any of the excipients in CERC-301.

### 4 Study Assessment by Visit

### 4.1 Screening (Day −28 to Day −1)

Patients completed a screening visit within 28 days of Visit 1. During the screening visit, the following procedures occurred.
1. Informed consent will be given and documented
2. Study eligibility (inclusion/exclusion) will be confirmed
3. Demographic information collected
4. Physical examination
5. Vital Signs
6. UPDRS Part 3
7. Patient Health Questionaire-9 (PHQ-9)
8. Orthostatic challenge test
9. Clinical safety laboratory samples will be collected
10. 12-lead electrocardiogram (ECG)
11. Medical history
12. Concomitant medications and adverse events will be recorded

Once patient eligibility was confirmed, Visit 1 was scheduled. Patients were instructed to discontinue treatment for their OH symptoms (midodrine, ephedrine, pyridostigmine) 72 hours prior to Visit 1.

### 4.2 Visits 1-5

The following procedures occurred at each visit. Study visits occurred between 7 and 10 days of the previous visit.
1. Confirmation that other OH medications have not been taken for at least 72 hours prior to the study visit
2. Randomization (Visit 1 and Visit 5 only)
3. Vital signs will be collected
4. PHQ-9
5. Clinical safety laboratory samples will be collected
6. Dosing will occur per randomized arm and visit
7. Concomitant medications and AEs will be recorded
8. An orthostatic challenge test (OST) will occur at pre-dose, 1, 2, 3, 4 and 6 hours (+/−15 min) post-dose. Patients were instructed not use supportive garments or compensatory mechanisms during the OST.
9. 12-lead ECG 1.5 hours (+/−15 minutes) post dose
10. nOH symptom (dizziness) assessment will occur following each orthostatic challenge

PK samples were drawn pre-dose and at 0.5, 1, 2, 4 and 6 hours post-dose. All blood draws were done after blood pressure measures are taken.

### 4.2 Follow-up (7-10 Days Following the Last Visit)

Subjects returned to the clinic 7-10 days after the last dose of study medication to collect any AEs and concomitant medications and administer the PHQ-9 and UPDRS.

### 5 Criteria for Evaluation

### 5.1 Safety Evaluations

### 5.1.1 Medical History, Demographic and Other Baseline Information

The medical history comprised:
General medical/surgical history
   Information collected includes condition/procedure, year of onset, and year ended or condition continuing
   For procedures and events such as accidents or fractures, year of occurrence should be entered for both year of onset and year ended
   History of tobacco use should be included in the medical history
   Medication history, including all medications (prescription, over-the-counter, and herbal medications) taken within the 6 weeks prior to Screening
Reproductive history for female subjects
   Post-menopausal, surgically sterile, or woman of childbearing potential (WOCBP)
   Date of last menses

The following demographic information were recorded:
Age (years) (based on date of birth and date of screening visit)
Ethnicity (Hispanic/Latino or not Hispanic/not Latino)
Race (White, American Indian/Alaska Native, Asian, Native Hawaiian or Other Pacific Islander, Black/African American, Other)
Gender (male or female)
Height (cm), without shoes
Body weight (kg), without shoes
Body Mass Index (BMI) (weight [kg]/height [m²]

Other baseline characteristics included:
Date of Parkinson's disease diagnosis
Date of nOH diagnosis
History of any drug or alcohol abuse

### 5.1.2 Adverse Events (AEs)

AEs were monitored and the following information recorded:
Verbatim complaint
Whether the event was treatment-emergent (TEAE)
Whether event was serious (SAE)
Date of onset
Severity of event
Relationship of event to study drug
Action taken regarding study drug due to the event
Clinical outcome of event (resolved or ongoing)
   If resolved, provide date of resolution

### 5.1.3 Vital Signs

The following vital signs were measured during the study:
Blood pressure (systolic and diastolic [mm Hg])
Heart rate (beats per minute [bpm])
Oral body temperature (°C)

Blood pressure and heart rate measurements were performed after the study subject had been seated and at rest minutes. Procedures for blood pressure measurement were detailed in the Site Operation Manual provided. When multiple procedures were planned at the same time point as blood pressure, blood pressure is measured first, followed by blood sampling (if applicable), and any other assessments.

Brachial arterial blood pressure were measured using an automated device, on the opposite arm to that from which blood samples are withdrawn. The same device were used throughout the study for each patient and measurements were always conducted on the same arm.

### 5.1.4 Clinical Laboratory Tests

Blood and urine samples were collected at each in-clinic study visit for routine clinical laboratory testing (hematology, chemistry, and urinalysis) and sent to a central laboratory for analysis. Additional, unscheduled testing were done during the study if medically indicated.

Any value outside the normal range was flagged for the attention of the Investigator or designee at the site. The Investigator or designee indicated whether or not the value is of clinical significance.

Laboratory parameters are outlined below. Blood sampling, volume collected, processing, storage, and shipping instructions are provided in the Laboratory Manual. Testing was performed by a central laboratory.
Hematology: The following hematology parameters were assessed: hemoglobin, hematocrit, red blood cell (RBC) count, white blood cell (WBC) count with differential, mean corpuscular volume (MCV), mean corpuscular hemoglobin (MCH), mean corpuscular hemoglobin concentration (MCHC), and absolute platelet count
Chemistry: The following clinical chemistry parameters were assessed: total protein, sodium, potassium, calcium, chloride, albumin, glucose, blood urea nitrogen (BUN), creatinine, uric acid, total bilirubin, alkaline phosphatase (AP), aspartate aminotransferase (AST), alanine aminotransferase (ALT), gamma glutamyl transferase (GGT), lactate dehydrogenase (LDH), creatine phosphokinase (CPK)
Urinalysis: Analyses for pH, glucose, ketones, specific gravity, nitrite, protein, bilirubin, and blood were performed. Microscopic urinalysis were performed if urinalysis results are abnormal
Pregnancy testing: Urine pregnancy test were utilized. If the urine pregnancy test was positive, a serum pregnancy test was conducted to confirm.
   5.1.5 12-lead Electrocardiograms

Standard safety 12-lead ECGs were performed during the study at approximately tₘₐₓ when applicable. The 12-lead ECGs were performed after the subject has been resting supine for ≥5 minutes. The ECG included all 12 standard leads and a Lead II rhythm strip on the bottom of the tracing. The ECG were recorded at a paper speed of 25 mm/sec. The following ECG parameters were collected: PR interval, QRS interval, RR interval, and QT interval. QTcB interval are calculated within the database from QT interval and RR interval.

$$QTcB = \frac{QT}{\sqrt{RR}}$$

All ECGs were evaluated by a qualified physician for the presence of abnormalities. Any clinically significant ECG abnormality were recorded as an AE.

### 5.1.6 7.1.6 Physical Examinations

The physical examination included an assessment of general appearance and a review of systems (dermatologic, head, eyes, ears, nose, mouth/throat/neck, respiratory, cardiovascular, gastrointestinal, extremities, musculoskeletal, and neurologic).

Any abnormality on physical examination that was not present at screening or worsened since screening were recorded as an AE.

Any abnormality on physical examination (including the unscheduled neurologic exam) that was not present at screening or worsened since screening were recorded as an AE.

### 5.1.7 Concomitant Medications

Site personnel documented any medications (over-the-counter [including herbal medications and vitamins] and prescription) the subject received within 6 weeks prior to screening and throughout the study. Recorded details included: medication name, start date and time, stop date and time, dose, route, frequency, and reason for use. The concomitant medication names were coded according to the WHO Drug Dictionary (WHODD) and classified by anatomical therapeutic chemical (ATC) categories.

### 5.1.8 Unified Parkinson's Disease Rating Scale (UPDRS)

The UPDRS Part 3 (Clinician scored monitored motor evaluation) was conducted at screening and Follow-up to determine any effects on motor function throughout the course of the study.

### 5.1.9 Patient Health Questionnaire—9 (PHQ-9)

Subjects completed the PHQ-9 at each study visit to ensure depression and the occurrence of suicidal thoughts were monitored.

### 5.2 Exploratory Evaluation

### 5.2.1 Blood Pressure

An exploratory evaluation of the effects of CERC-301 on blood pressure in subjects with nOH was conducted. Each subject completes an orthostatic challenge test as outlined in Table 5.

SBP, DBP, and heart rate were measured in the seated position, then recumbent (head elevated to 30 degrees) at −10, −5 minutes, immediately prior to standing (0 time point) and then at 1, 3, and 5 minutes in a standing position, followed by a final seated position measurement, as shown in Table 5.

TABLE 5
Order and timing of BP and heart rate measurements

| | | Recumbent position* | | | | Standing | | | |
|---|---|---|---|---|---|---|---|---|---|
| | | −10 | −5 | Immediately | Stand | +1 | +3 | +5 | |
| | Seated | min | min | prior | 0 min | min | min | min | Seated |
| Systolic and | √ | √ | √ | √ | x | √ | √ | √ | √ |
| diastolic BP, | | | | | | | | | |
| and heart rate | | | | | | | | | |

*Subjects did not lay flat. Measurements in the supine position were recorded with the subjects' head elevated to 30 degrees.

If the investigator considered that a patient was at immediate risk of fainting at any time during the standing test, a blood pressure measurement was obtained before the patient was returned to a supine position, when possible. The time standing occurred (achieving an upright positions on their feet), the blood pressure and the time that it was measured at was recorded on the case report form (CRF).

Brachial arterial blood pressure was measured using an automated device on the opposite arm to that from which blood samples are drawn. The same device was used throughout the study for each patient and measurements were always be conducted on the same arm. The investigator or their designee did not inform patients of their orthostatic blood pressure measurements during the study. Patients were asked to empty their bladder before the blood pressure and heart rate assessments were conducted.

In addition to the blood pressures collected during the orthostatic challenge test, vital signs were collected at every visit for safety. Blood pressure was taken at approximately the same time during each visit and from the same arm after a minimum of 5 minutes resting in the seated position.

5.2.2 Symptomatic assessment

Symptomatic assessment was conducted using a modified version of Item #1 of the Orthostatic Hypotension Symptom Assessment (OHSA). Item #1 of the OHSA measures the level of “Dizziness/Lightheadedness/Feeling Faint/or Feeling Like You Might Blackout” a subject feels over the course of a one-week period. OHSA Item #1 is a Likert scale that ranges from 0 (none) through 10 (worst). For this study, a modification to Item #1 asked subjects to report their level of “Dizziness/Lightheadedness/Feeling Faint/or Feeling Like You Might Blackout” acutely during orthostatic challenge at various time points before and after dosing. If the subject blacked out during the orthostatic challenge, the score was recorded as a “10.”

### 5.3 Pharmacokinetic Evaluation

Blood samples for plasma concentration analysis of CERC-301 were collected at pre-dose and 0.5, 1, 2, 4 and 6 hours post-dose at all visits.

Blood sampling, processing, storage, and shipping instructions were provided in the Laboratory Manual. The plasma samples were analyzed via liquid chromatography tandem mass spectrometry (LC-MS/MS) following a validated method.

### 5.4 Statistical Methods

### Data Presentation/Descriptive Statistics

A Statistical Analysis Plan was completed prior to the interim analysis and outlined all relevant parameters and analyses for the interim as well as the end of study analyses.

In general, summary statistics (n, mean, standard deviation, median, minimum, maximum, and, for all continuous parameters, the coefficient of variation) is presented.

### Sample Size Considerations

Formal sample size calculations were not performed. The number of subjects was chosen based on feasibility and is considered sufficient to meet the study objectives.

### Analysis Sets

Two analysis sets were used. The safety analysis set (SAF) was the primary analysis set for all safety displays. The SAF consists of all randomized subjects who receive at least one dose of study medication. The PK analysis set (PKAS) is the primary analysis set for all PK displays and analyses. The PKAS consists of all subjects from the SAF for whom sufficient plasma concentration data are available to facilitate the calculation of at least one PK parameter.

### Safety and Tolerability

All safety and tolerability data are summarized using descriptive statistics. They are listed and summarized in tabular and/or graphical form. No formal statistical testing were performed on these data. Descriptive statistics for laboratory values and their change or shift from baseline for each dose group are provided for each scheduled time of collection. Descriptive statistics for vital signs and their change from baseline for each dose group are provided for each scheduled time of collection. The placebo subjects are combined into a single placebo group for summary presentations.

### Pharmacokinetics

PK parameter estimates for CERC 301 were calculated using standard non-compartmental methods of analysis.

### 6 Results

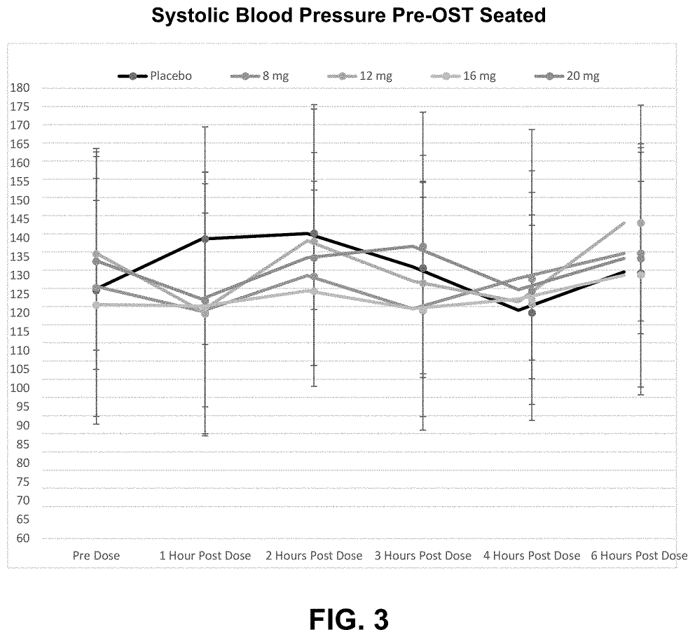

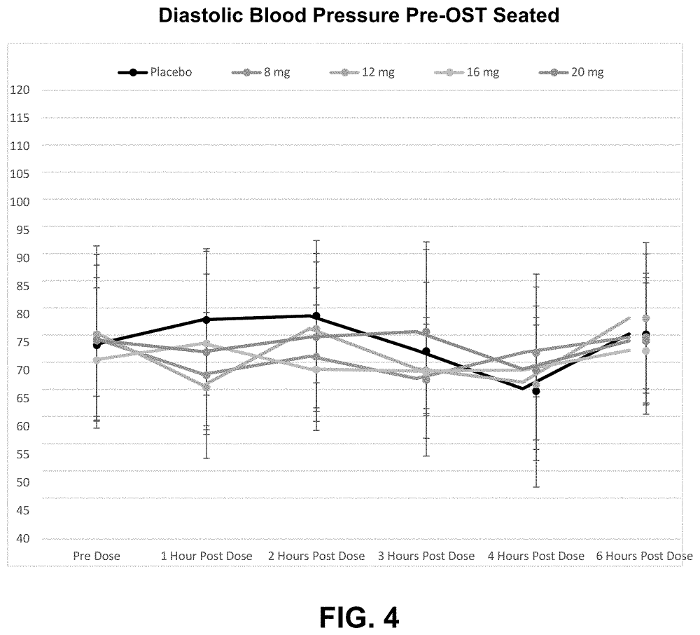

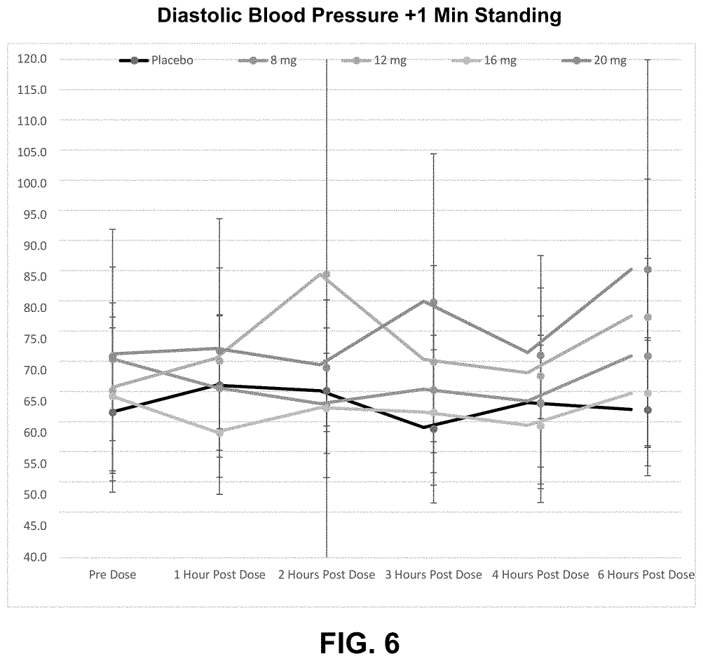

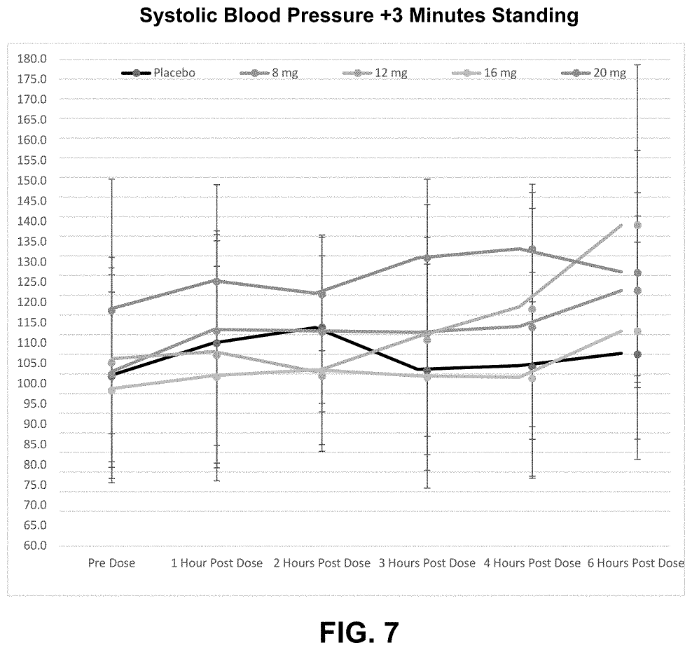

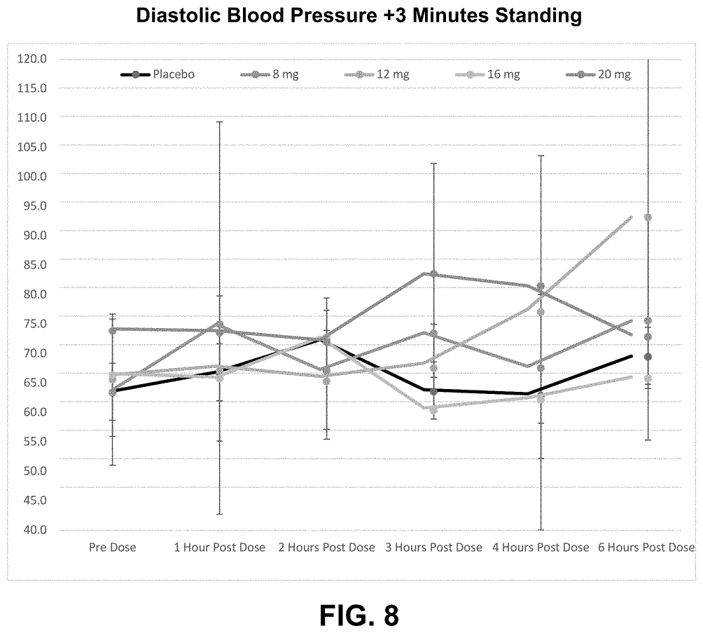

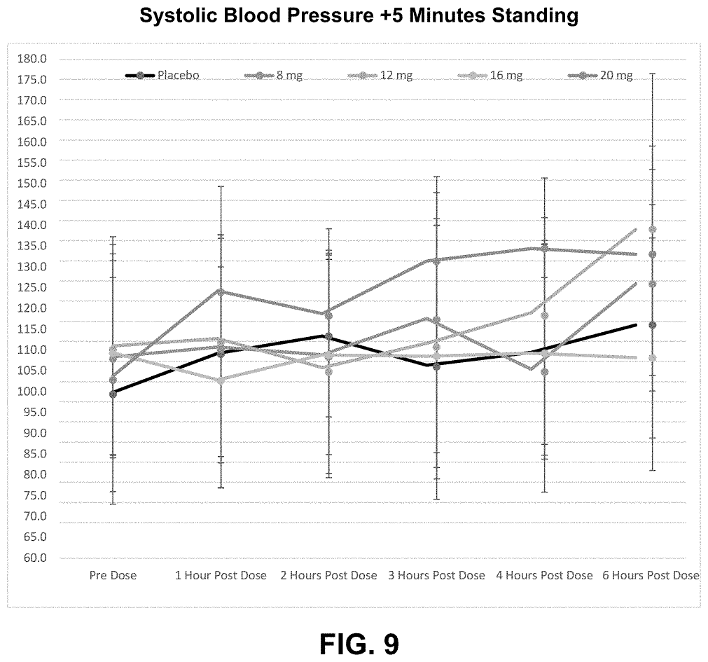

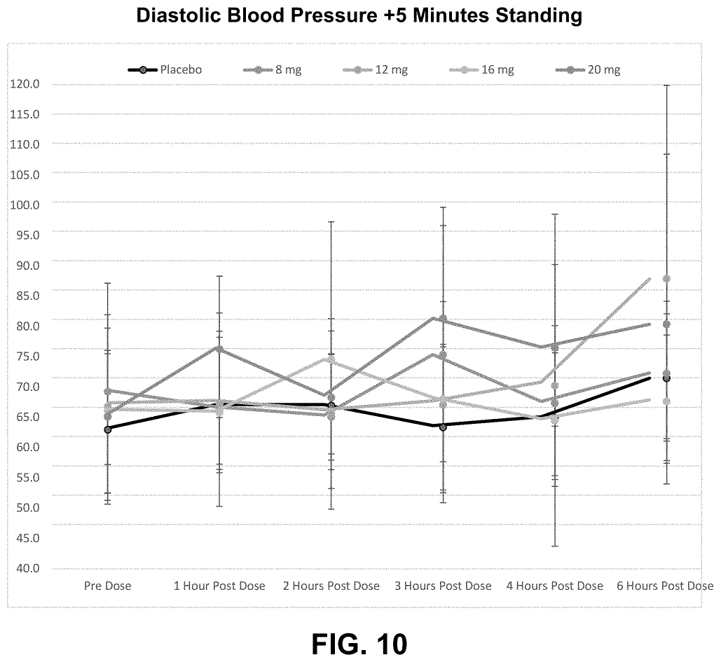

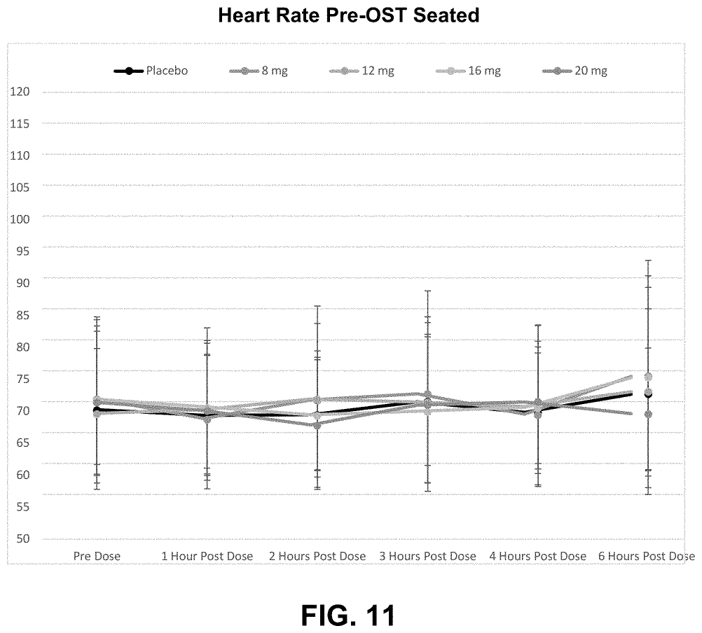

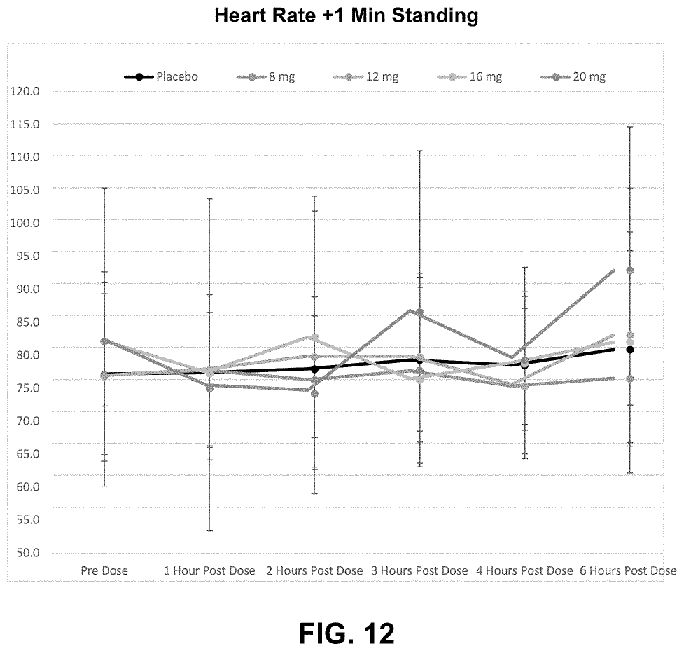

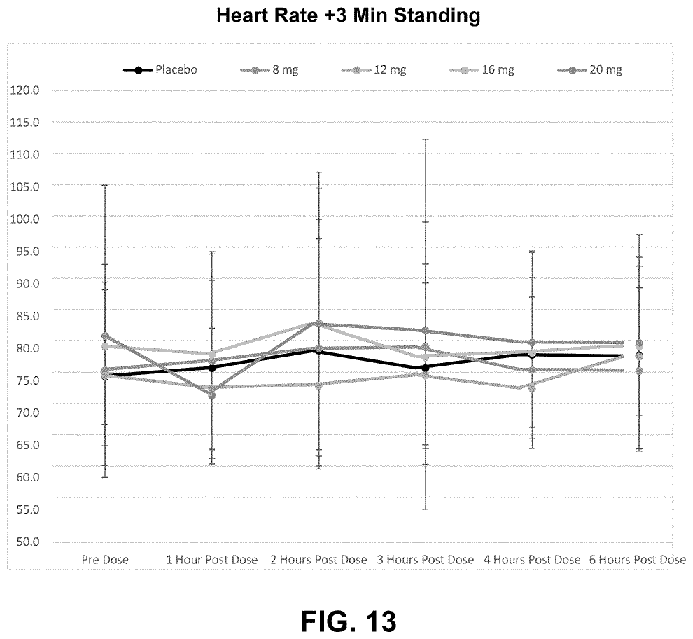

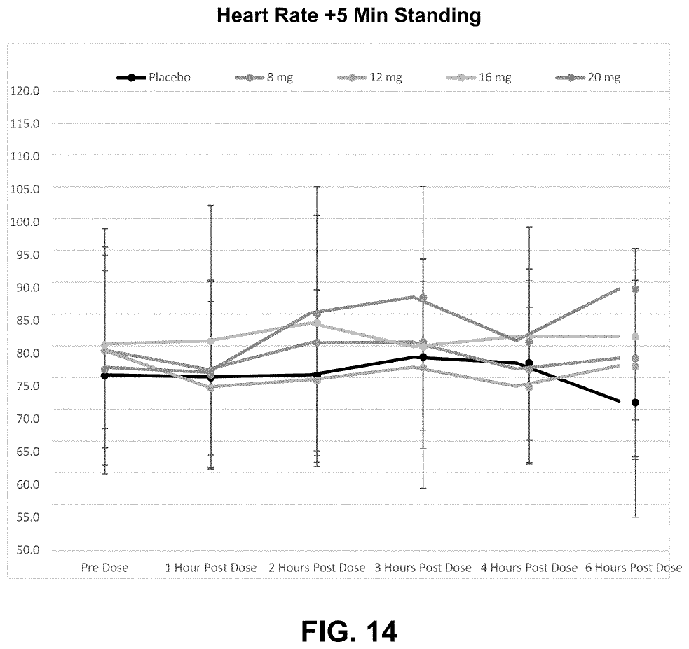

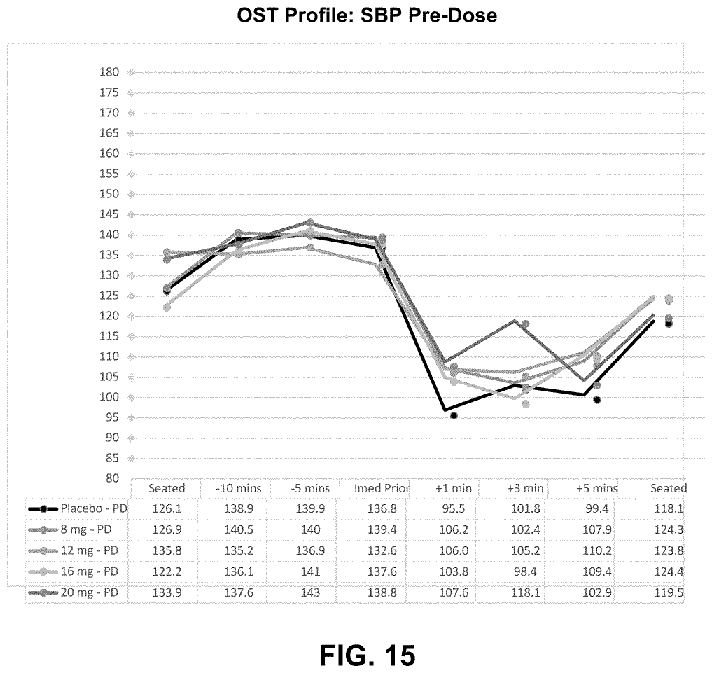

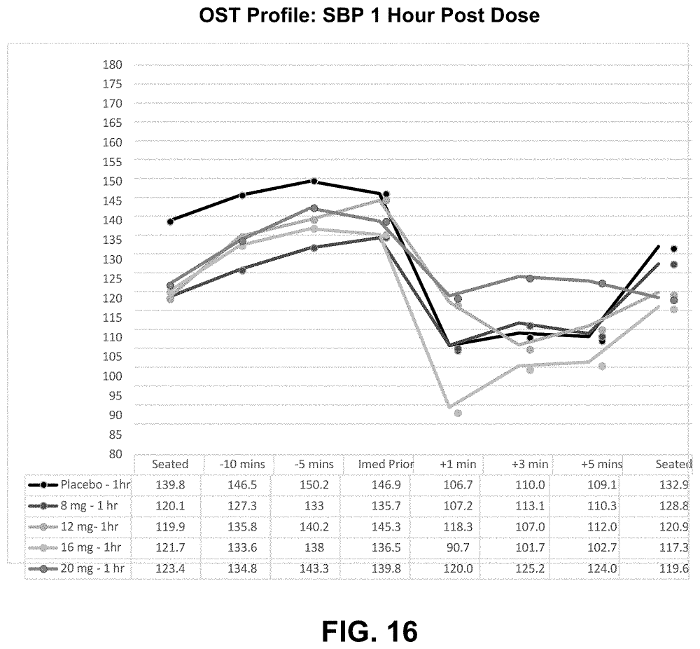

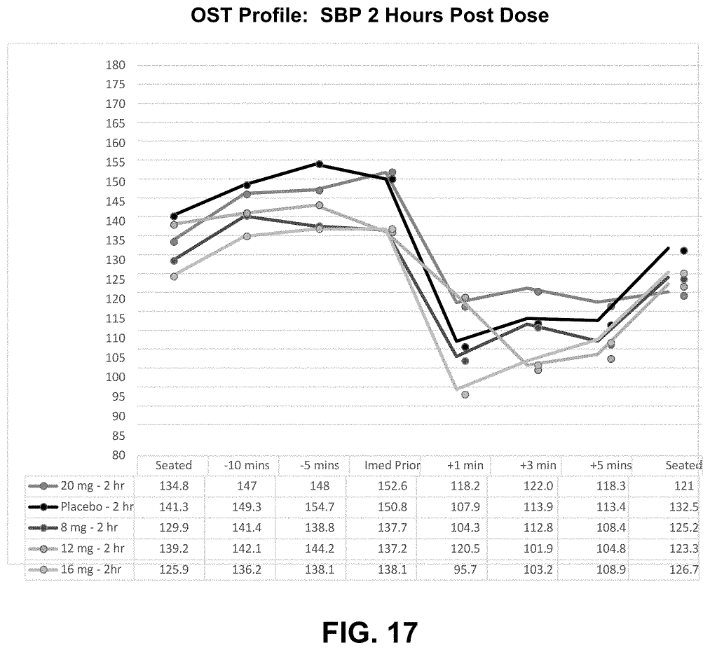

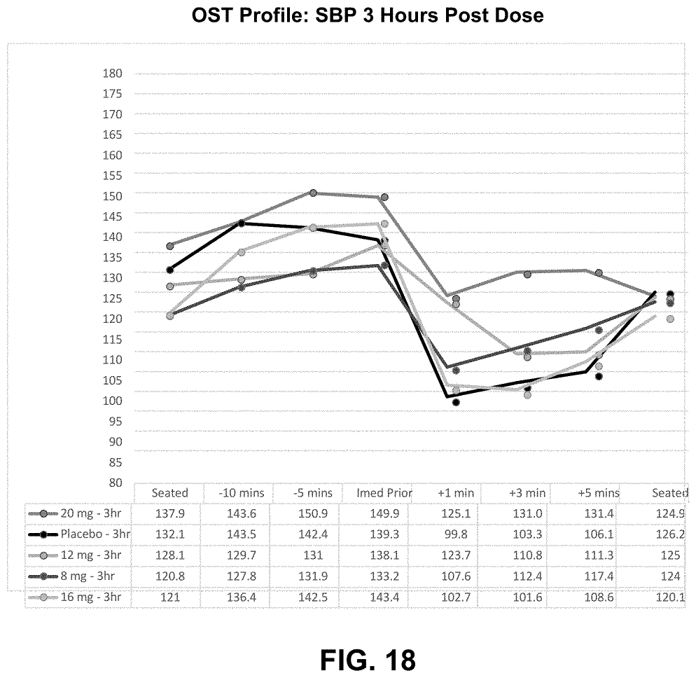

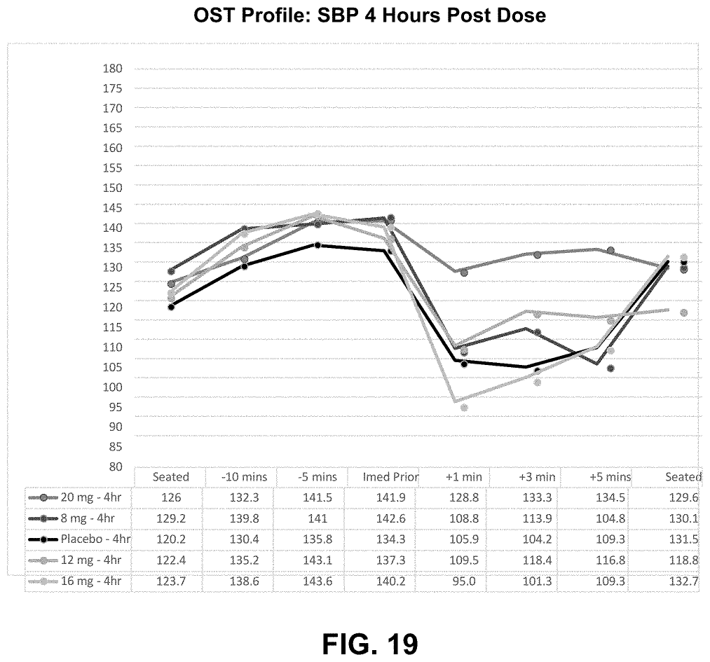

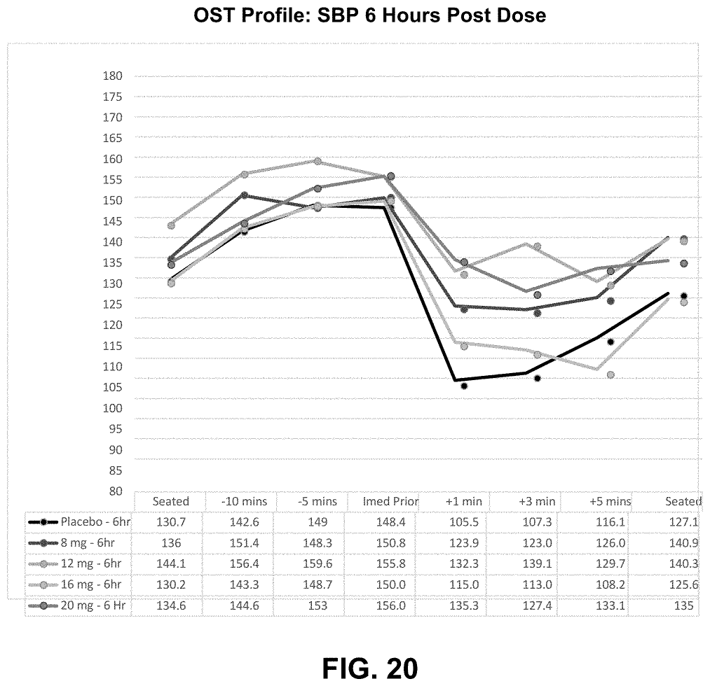

FIGS. 3-20 graphically show the effects of four different doses of CERC-301 (8 mg, 12 mg, 16 mg and 20 mg P.O.) compared to placebo on systolic and diastolic blood pressure, heart rate and symptomatic results during the orthostatic challenge test at the protocol defined timepoints.

### Safety and Tolerability

Single doses of CERC-301 at 8 mg, 12 mg, 16 mg and 20 mg were considered safe and well tolerated in patients with nOH associated with Parkinson's disease. No signals of increased heart rate or, supine hypertension during the recumbent phase of the OST, were observed between active treatment and placebo.

No SAEs were reported. Nine subjects reported a total of 16 AEs. AEs reported were dizziness (7.1%), hypoaesthesia (7.1%), memory impairment (7.1%), on/off phenomenon (7.1%), somnolence (7.1%), blurred vision (14.3%), fall (14.3%), squamous cell carcinoma (7.1%), nasal congestion (7.1%), and hypotension (7.1%).

### Pharmacokinetics

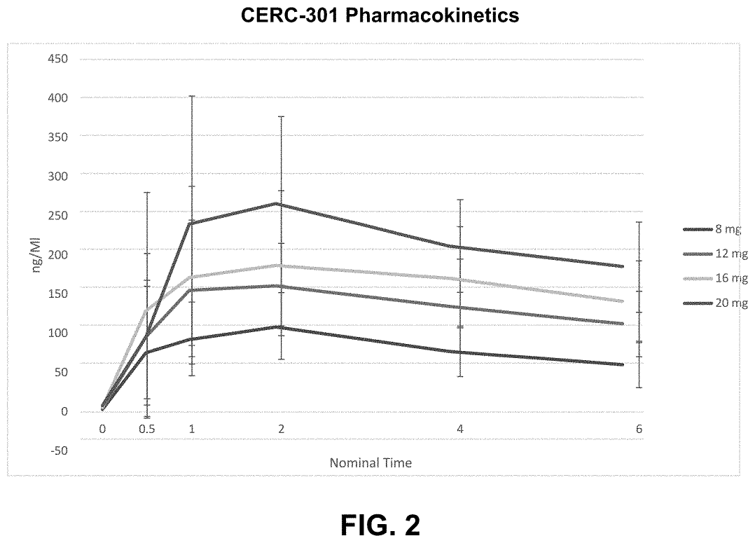

Pharmacokinetic samples were drawn at pre-dose, 0.5, 1, 2, 4 and 6 hours post dose. As shown in FIG. 2, Cₘₐₓ was generally dose proportional between each dosing group. Tmax was observed at approximately 2 hours post dose for all dosing groups.

Peak plasma concentration were seen at approximately 2 hours with Cmax being generally dose proportional at 108.5 ng/mL, 160.9 ng/mL, 187.0 ng/mL, and 265.78 ng/mL for doses of 8 mg, 12 mg, 16 mg, and 20 mg, respectively. Area Under the Curve (AUC) was 473.0 ng*hr/mL, 794.9 ng*hr/mL, 954.2 ng*hr/mL, and 1239.8 ng*hr/mL for doses of 8 mg, 12 mg, 16 mg and 20 mg, respectively over the 6-hour PK collection period. Elimination half-life and AUC (0-infin) were not calculated due to the limited number of PK samples taken. Effects of CERC-301 on Symptomatic Orthostatic Hypotension

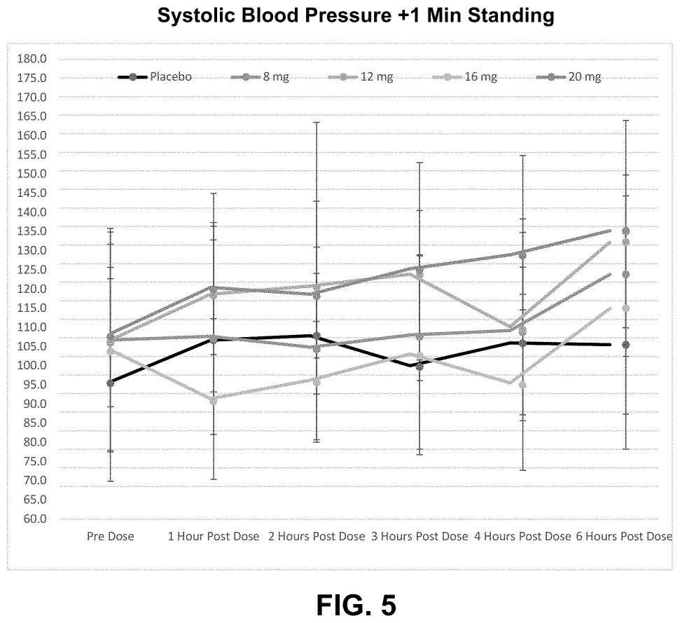

As shown in FIGS. 5-10 and FIGS. 15-20, improvements in standing blood pressure were seen at all doses out to and including the 6-hour post dose timepoint, with the 20 mg dose showing the most robust improvement overall compared to placebo.

### 7 Conclusion

The data presented herein suggest that selective NR2B antagonists such as CERC-301 may provide a long-term therapeutic treatment of symptomatic orthostatic hypotension that is well tolerated and effective.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

### REFERENCES

¹ Goldstein D S, et al. (2002). Orthostatic hypotension from sympathetic denervation in Parkinson's disease. Neurology 58(8): 1247-1255.
² Kaufmann H, and Biaggioni I (2003). Autonomic failure in neurodegenerative disorders. Semin. Neurol. 23(4): 351-363.
³ Metzler, M et al. (2013). Neurogenic orthostatic hypotension: pathophysiology, evaluation, and management. J Neurol. Vol 260 (9): 2212-2219.
⁴ Kaufmann, H, et al. (2017). Neurogenic orthostatic hypotension: the very basics. Clin. Auton. Res. 27 (suppl 1): 39-43.
⁵ Freeman, R (2008). Neurogenic Orthostatic Hypotension. N. Engl. J. Med. 358: 615-624.
⁶ Low, P A and Wolfgang Singer, W (2008). Update on Management of Neurogenic Orthostatic Hypotension. Lancet Neurol. 2008 May; 7(5): 451-458.
⁷ Norcliffe-Kaufmann L, et al. (2013). Developmental abnormalities, blood pressure variability and renal disease in Riley Day syndrome. J. Hum. Hypertens. 27(1): 51-55.
⁸ Kaufmann H, et al. (1988). Treatment of orthostatic hypotension due to autonomic failure with a peripheral alpha-adrenergic agonist (midodrine). Neurology 38(6): 951-956.
⁹ ProAmatine (midodrine hydrochloride) [package insert]. 2017.
¹⁰ Kaufmann H, et al. (2015). Droxidopa in neurogenic orthostatic hypotension. Expert Rev. Cardiovasc. Ther. 13(8): 875-891.
¹¹ Northera (droxidopa) [package insert]. 2014
¹² Northera (droxidopa) [package insert]. 2014
¹³ Metzler, M et al. (2013). Neurogenic orthostatic hypotension: pathophysiology, evaluation, and management. J Neurol. 260 (9): 2212-2219.
¹⁴ Metzler, M et al. (2013). Id.
¹⁵ Kaufmann, H et al. (2017). Neurogenic orthostatic hypotension: the very basics. Clin. Auton. Res. 27 (suppl 1): 39-43.
¹⁶ Freeman, R. (2008). Neurogenic Orthostatic Hypotension. N Engl J Med. 358: 615-624.
¹⁷ Freeman R, et al. (2011). Consensus statement on the definition of orthostatic hypotension, neurally mediated syncope and the postural tachycardia syndrome. Clin Auton Res. 21:69-72.
¹⁸ Freeman R, et al. (2011). Id.
¹⁹ Low P A. (2008). Prevalence of orthostatic hypotension. Clin Auton Res. 18(Suppl 1): 8-13.
²⁰ Goldstein D S and Sharabi Y (2009). Neurogenic orthostatic hypotension: a pathophysiological approach. Circulation 119:139-146.
²¹ Marti N M, et al. (2003). Clinical overview of the synucleinopathies. Mov Disord. 18(Suppl 6): S21-S27.
²² Senard J M, et al. (1997). Prevalence of orthostatic hypotension in Parkinson's disease. J. Neurol. Neurosurg. Psychiatry 63: 584-589.
²³ Wood B H, et al. (2002). Incidence and prediction of falls in Parkinson's disease: a prospective multidisciplinary study. J. Neurol. Neurosurg. Psychiatry 72: 721-725.
²⁴ Allan L M, et al. (2007). Autonomic dysfunction in dementia. J. Neurol. Neurosurg. Psychiatry 78: 671-677.
²⁵ Sonnesyn H, et al. (2009). High prevalence of orthostatic hypotension in mild dementia. Dement. Geriatr. Cogn. Disord. 28: 307-313.
²⁶ Thaisetthawatkul P, et al. (2004). Autonomic dysfunction in dementia with Lewy bodies. Neurology 62: 1804-1809.
²⁷ Braune S, et al. (1999). Lucking CH. Cardiac uptake of [¹²³1]MIBG separates Parkinson's disease from multiple system atrophy. Neurology 53: 1020-1025.
²⁸ Hanyu H, et al. (2006). Comparative value of brain perfusion SPECT and [(¹²³)1]MIBG myocardial scintigraphy in distinguishing between dementia with Lewy bodies and Alzheimer's disease. Eur. J. Nucl. Med. Mol. Imaging 33: 248-253.
²⁹ Kollensperger M, et al. (2010). Presentation, diagnosis, and management of multiple system atrophy in Europe: final analysis of the European multiple system atrophy registry. Mov. Disord. 25: 2604-2612.
³⁰ Kaufmann H, et al. (2001). Accumulation of alpha-synuclein in autonomic nerves in pure autonomic failure. Neurology 56: 980-981.
³¹ Shibao C, et al. (2013). ASH position paper: evaluation and treatment of orthostatic hypotension. J. Clin. Hypertens. 15(3): 147-153.
³² Schroeder C, et al. (2013). Management of neurogenic orthostatic hypotension in patients with autonomic failure. Drugs 73(12): 1267-1279.
³³ Low, PA and Wolfgang Singer, W (2008). Update on Management of Neurogenic Orthostatic Hypotension. Lancet Neurol. 7(5): 451-458.
³⁴ Norcliffe-Kaufmann L, et al. (2013). Developmental abnormalities, blood pressure variability and renal disease in Riley Day syndrome. J. Hum. Hypertens. 27(1):51-55.
³⁵ Kaufmann H, et al. (1988). Treatment of orthostatic hypotension due to autonomic failure with a peripheral alpha-adrenergic agonist (midodrine). Neurology 38(6): 951-956.
³⁶ ProAmatine (midodrine hydrochloride) [package insert]. 2017.
³⁷ Kaufmann H, et al. (2015). Droxidopa in neurogenic orthostatic hypotension. Expert Rev. Cardiovasc. Ther. 13(8): 875-891.
³⁸ Northera (droxidopa) [package insert]. 2014
³⁹ Northera (droxidopa) [package insert]. 2014
⁴⁰ Ibrahim L, et al. (2012). A Randomized, placebo-controlled, crossover pilot trial of the oral selective NR2B antagonist MK-0657 in patients with treatment-resistant major depressive disorder. J. Clin. Psychopharmacol. 32(4): 551-557.
⁴¹ Addy C, et al. (2009). Single-dose administration of MK-0657, an NR2B-selective NMDA antagonist, does not result in clinically meaningful improvement in motor function in patients with moderate Parkinson's disease. J. Clin. Pharmacol. 49(7): 856-64.
⁴² Addy C, et al. (2009). Id.

All references disclosed herein are incorporated by reference in their entirety.

1. A method for treating symptomatic orthostatic hypotension in a human patient, the method comprising administering to the patient a pharmaceutical composition comprising an effective amount of a NR2B antagonist.

2. The method of claim 1, wherein administration of the compound to the patient results in one or more of: (a) an increase in the patient's seated systolic blood pressure; (b) an increase in the patient's standing time; and (c) a decrease in dizziness or lightheadedness experienced by the patient.

3. The method of claim 1, wherein the symptomatic orthostatic hypotension is neurogenic orthostatic hypotension.

4. The method of claim 1, wherein the patient suffers from a neurodegenerative disease selected from the group consisting of: multiple system atrophy, pure autonomic failure, dementia with Lewy bodies, and Parkinson's disease.

5. The method of claim 1, wherein the patient has Parkinson's disease.

6. A method for treating symptomatic orthostatic hypotension and the symptoms thereof in a human patient, the method comprising administering to the patient a pharmaceutical composition comprising an effective amount of Compound (I):

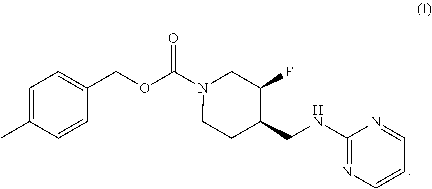 [SMILES: Cc1ccc(COC(=O)N2CC[C@H](CNc3ncccn3)[C@H](F)C2)cc1]

7. The method of claim 6, wherein the effective amount of Compound (I) is an amount ranging from about 0.5 mg/day to about 50 mg/day.

8. The method of claim 6, wherein the effective amount of Compound (I) is an amount ranging from about 5.0 mg/day to about 20 mg/day.

9. The method of claim 6, wherein the effective amount of Compound (I) is a dose selected from the group consisting of: 8.0 mg/day, 12 mg/day, 16 mg/day, and 20 mg/day.

10. The method of claim 6, wherein Compound (I) is the crystalline form of Compound (I).

11. The method of claim 6, wherein administration of Compound (I) to the patient results in one or more of: (a) an increase in the patient's seated systolic blood pressure; (b) an increase in the patient's standing time; and (c) a decrease in dizziness or lightheadedness experienced by the patient.

12. The method of claim 6, wherein the patient suffers from a neurodegenerative disease selected from the group consisting of:
multiple system atrophy, pure autonomic failure, dementia with Lewy bodies, and Parkinson's disease.

13. The method of claim 6, wherein the patient has Parkinson's disease.

14. The method of claim 6, wherein the symptomatic orthostatic hypotension is neurogenic orthostatic hypotension.

15. The method of claim 6, wherein Compound (I) is administered with an agent selected from an al-adrenoceptor agonist, an α-2 adrenergic receptor antagonist, a corticosteroid, a norepinephrine precursor, and a cholinesterase inhibitor, or a combination thereof.

16. The method of claim 6, wherein Compound (I) is administered with midodrine, fludrocortisone acetate, droxidopa or pyridostigmine, or, in each case, a pharmaceutically-acceptable salt thereof.

17. The method of claim 6, wherein Compound (I) is administered with midodrine hydrochloride, fludrocortisone acetate, droxidopa or pyridostigmine bromide.
